(19) United States
(12) Patent Application Publication
(10) Pub. No.: US 2023/0305023 A1
(43) Pub. Date: Sep. 28, 2023

(54) METHODS OF TREATMENT AND DIAGNOSTIC OF PATHOLOGICAL CONDITIONS ASSOCIATED WITH INTENSE STRESS

(71) Applicants: INSERM (INSTITUT NATIONAL DE LA SANTÉ ET DE LA RECHERCHE MÉDICALE), Paris (FR); UNIVERSITÉ DE BORDEAUX, Bordeaux (FR)

(72) Inventors: Jean-Michel REVEST, Bordeaux (FR); Aline DESMEDT, Bordeaux (FR); Monique VALLEE, Bordeaux (FR); Pier Vincenzo PIAZZA, Bordeaux (FR)

(21) Appl. No.: 18/003,274

(22) PCT Filed: Jun. 24, 2021
(86) PCT No.: PCT/EP2021/067397

§ 371 Date: Dec. 23, 2022

(30) Foreign Application Priority Data
Jun. 25, 2020 (EP) 20305702.1

(51) Int. Cl. G01N 33/68; C07K 16/38; A61K 31/713

(52) U.S. Cl. CPC G01N 33/6893; A61K 31/713; C07K 16/38; G01N 2333/4704; G01N 2333/745; G01N 2800/30; G01N 2800/50; G01N 2800/52

(57) ABSTRACT

The present invention relates to a method for preventing or treating pathological conditions associated with intense stress such as Post-Traumatic Stress Disorder (PTSD) by targeting the endogenous PAI-1 (Type 1 Plasminogen Activator Inhibitor). In the present invention, inventors demonstrate that there is a shift in the balance between the expression of tPA and PAI-1 proteins in a hippocampal region of a preclinical model of Post-Traumatic Stress (PTSD), is responsible for the transition between moderate stress which increases memory and facilitates adaptation and intense stress intense stress which induce pathological memories. In conditions of moderate stress, glucocorticoid hormones (GC) increase the expression of the tPA protein in the hippocampal brain region which by triggering the Erk1/2^{MAPK} cascade strengthens memory. When stress is particularly intense, very high levels of GC then increase the production of PAI-1 protein, which by blocking the activity of tPA induces PTSD-like memories. Accordingly, inhibition of PAI-1 activity represent a new therapeutic approach to this debilitating condition and PAI-1 body fluid level in patient after trauma could be a predictive biomarker of the subsequent appearance of PTSD.

### FIELD OF THE INVENTION

The present invention relates to a method for preventing or treating pathological conditions associated with intense stress such as Post-Traumatic Stress Disorder (PTSD) by targeting the endogenous PAI-1 (Type 1 Plasminogen Activator Inhibitor). The present invention also relates to a method for diagnosis pathological conditions associated with intense stress, such as Post-Traumatic Stress Disorder (PTSD) by detecting the serum level of endogenous PAI-1.

### BACKGROUND OF THE INVENTION

Stressful events trigger a set of biological responses which generally increase adaptation to potentially harmful situations. However, overly intense or chronic stress can have deleterious effects leading to several behavioral disorders including Substance Use Disorders (SUD), depressive-like and anxiety-like disorders, in particular Post-Traumatic Stress Disorder (PTSD) (1-4). Memory performances are a prototypical example of this dichotomy between the beneficial and pathological effects of stress. Moderate stress increases the memory of associated events facilitating adaptation to future similar situations (2;3). In contrast, intense stress can alter memory consolidation leading to pathological conditions such as PTSD (3-6). PTSD as a severe stress-related disorder is developed by 10 to 20% of subjects experiencing a strong traumatic life event (e.g. rapes, terrorist attacks or military fights) (7;8). PTSD is characterized by recurrent and intrusive recollections of the trauma due to the inability of the individual to restrict fear to the appropriate predictor of the threat (5;7;9). In humans, it was shown to be associated with a hippocampal dysfunction that might contribute to the deficit of contextual memory of the trauma (9-11). This progressive shift from adaptive to deleterious consequences as a function of stress intensity follows an inverted-U pattern and has been known since the beginning of the twentieth century (12;13), but the molecular mechanisms of this pathophysiological process remain largely unknown.

Glucocorticoid hormones (GC), one of the major biological responses to stress, have been proposed as one of the factors involved in the shift from beneficial to pathological effects of stress. The increase in GC induced by moderate stressors enhances the memory of stress-associated events (14-16) whilst a further increase in GC concentration can induce PTSD-like memories (5). Both effects of GC are mediated by the activation in the brain of the glucocorticoid receptor (GR), a hormone-activated transcription factor belonging to the family of nuclear receptors (17). In condition of moderate stress, the activation of the GR in the hippocampus, one of the major brain structures involved in memory processing and in PTSD pathophysiology (10), induces a cascade of molecular events that increases memory encoding. The first step is the increase of tPA (tissue Plasminogen Activator) which cleaves the pro-BDNF to mature BDNF. Mature BDNF, by activating the TrkB receptor, phosphorylates Erk1/2^{MAPK} which, increasing the expression of the downstream transcription factor Egr-1, finally enhances the levels of memory-enhancing effector proteins, such as Synapsin-Ia/Ib (14-16). In this report inventors showed that a deregulation of this signaling pathway in the dorsal hippocampus triggered by an increase in GC-induced PAI-1 (Type 1 Plasminogen Activator Inhibitor) levels underlies the transition from a normal to PTSD-like fear memory.

### SUMMARY OF THE INVENTION

A first object of the invention relates to PAI-1 antagonist for use in the prevention or treatment of pathological conditions associated with intense stress selected from the list consisting of Substance Use Disorders (SUD), depressive-like and anxiety-like disorders and Post-Traumatic Stress Disorder (PTSD).

In a particular embodiment, the pathological conditions associated with intense stress is Post-Traumatic Stress Disorder (PTSD).

A second object of the invention relates to a method for diagnosis a pathological condition associated with intense stress, selected from the list consisting of Substance Use Disorders (SUD), depressive-like and anxiety-like disorders, in particular Post-Traumatic Stress Disorder (PTSD) by detecting the serum level of endogenous PAI-1.

### DETAILED DESCRIPTION OF THE INVENTION

Here the inventors investigated the link between the PAI-1 level of expression at hippocampus brain region and pathological conditions associated with intense stress as observed in Post-Traumatic Stress (PTSD). Moderate stress increases memory and facilitates adaptation. In contrast, intense stress can induce pathological memories as observed in Post-Traumatic Stress Disorders (PTSD). In the present study, inventors shows that a shift in the balance between the expression of tPA and PAI-1 proteins is responsible for this transition. In conditions of moderate stress, glucocorticoid hormones (GC) increase the expression of the tPA protein in the hippocampal brain region which by triggering the Erk1/2^{MAPK} cascade strengthens memory. When stress is particularly intense, very high levels of GC then increase the production of PAI-1 protein, which by blocking the activity of tPA induces PTSD-like memories. Accordingly, PAI-1 levels after trauma could be a predictive biomarker of the subsequent appearance of PTSD and pharmacological inhibition of PAI-1 activity a new therapeutic approach to this debilitating condition.

### Therapeutic Methods and Uses

The present invention provides methods and compositions (such as pharmaceutical compositions) for preventing or treating a pathological conditions associated with intense stress selected from the list consisting of Substance Use Disorders (SUD), depressive-like and anxiety-like disorders, in particular Post-Traumatic Stress Disorder (PTSD). In the context of the invention, the term “treatment or prevention” means reversing, alleviating, inhibiting the progress of, or preventing the disorder or condition to which such term applies, or one or more symptoms of such disorder or condition. In particular, the treatment of the disorder may consist in reducing the number of pathological memories as observed in Post-Traumatic Stress Disorders (PTSD). Most preferably, such treatment leads to the complete depletion of the pathological memories as observed in Post-Traumatic Stress Disorders (PTSD). Preferably, the individual to be treated is a human or non-human mammal (such as a rodent a feline, a canine, or a primate) affected or likely to be affected with pathological memories as observed in Post-Traumatic Stress Disorders (PTSD).

Preferably, the individual is a human.

According to a first aspect, the present invention relates to a PAI-1 antagonist for use in the prevention or the treatment of a patient affected with a pathological conditions associated with intense stress selected from the list consisting of Substance Use Disorders (SUD), depressive-like and anxiety-like disorders, in particular Post-Traumatic Stress Disorder (PTSD).

As used herein the term “PAI-1” (Plasminogen Activator Inhibitor-1) also known as “endothelial plasminogen activator inhibitor” or serpin E1, has its general meaning in the art. PAI-1 is a serine protease inhibitor and is the principal inhibitor of tissue plasminogen activator (tPA) and urokinase (uPA) the activators of plasminogen in plasmin and hence fibrinolysis (which refers to the degradation of fibrin within a blood clot). PAI-1 inhibits their target proteases by a substrate suicide mechanism that forms an initial noncovalent Michaelis-like complex, an acyl intermediate, and finally a stable covalent complex. This direct interaction is terminal, inactivating both the protease and the inhibitor. Additional inhibition is mediated by PAI-1 binding to the uPA/uPA receptor complex. Accordingly, PAI-1 binds to and inactivates free uPA, as well as uPA that is bound to uPAR. The PAI-1-uPA-uPAR complex, but not the uPA-PAI-1 complex, is internalized by the LRP receptor, which decreases uPA induced cell migration (Degryse B, et al. FEBS Lett 2001;505:249-54). In plasma, PAI-1 exists in two major forms: active and latent. Active PAI-1 is able to effectively inhibit target proteases, while latent PAI-1 is inactive. Active PAI-1 is also unstable and spontaneously converts into its latent form within 2 h of exposure to temperatures of at least 37° C. (Lindahl TL, et al. Thromb Haemost 1989;62:748-51). To prevent this conversion from transpiring, PAI-1 binds to the ECM protein, Vitronectin (VN). VN does not bind to inactive PAI-1 or PAI-1 in complex with its target proteases and in vivo VN stabilizes PAI-1 at least two- to threefold (Declerck PJ, et al. J Biol Chem 1988;263:15454-61). It has been shown that VN protects PAI-1 from inhibition, since the efficacy of several inhibitors diminishes in its presence (Elokdah H, et al J Med Chem 2004;47:3491-4; and Gorlatova NV, et al.. J Biol Chem 2007;282:9288-96).

In humans PAI-1 protein is encoded by the SERPINE1 gene located on chromosome 7 (7q21.3-q22 / Gene ID: 5054). One example of wild-type PAI-1 human amino acid sequence is provided in SEQ ID NO:1 (UniProtKB P05121/ NCBI Reference Sequence: NP_000593). One example of nucleotide sequence encoding wild-type human PAI-1 is provided in SEQ ID NO:2 (NCBI Reference Sequence: NM_NM_000602).

Of course variant sequences of the PAI-1 may be used in the context of the present invention, those including but not limited to functional homologues, paralogues or orthologues of such sequences.

A “PAI-1 antagonist” refers to a molecule (natural or synthetic) capable of neutralizing, blocking, inhibiting, abrogating, reducing or interfering with the biological activities of PAI-1 including, for example, reduction or blocking the interaction for instance between PAI-1 and tPA or uPA. PAI-1 antagonists include antibodies and antigen-binding fragments thereof, proteins, peptides, glycoproteins, glycopeptides, glycolipids, polysaccharides, oligosaccharides, nucleic acids, bioorganic molecules, peptidomimetics, pharmacological agents and their metabolites, transcriptional and translation control sequences, and the like. Antagonists also include, antagonist variants of the protein, siRNA molecules directed to a protein, antisense molecules directed to a protein, aptamers, and ribozymes against a protein. For instance, the PAI-1 antagonist may be a molecule that binds to PAI-1 and neutralizes, blocks, inhibits, abrogates, reduces or interferes with the biological activity of PAI-1 (such as inhibition of signaling pathway tPA/TrkB/Erk1/2^{MAPK} in hippocampal region).

More particularly, the PAI-1 antagonist according to the invention is:
1) an inhibitor of PAI-1 activity (such as small organic molecule, antibody, aptamer, polypeptide) and/ or
2) an inhibitor of PAI-1 gene expression (such as antisense oligonucleotide, nuclease, siRNA, ...)

By “biological activity” of PAI-1 is meant in the context of the present invention, inducing PTSD-like memories (through blocking the tPA activity regarding the pro-mnesic tPA/BDNF/TrkB/Erk1/2^{MAPK} signaling cascade)

Tests for determining the capacity of a compound to be a PAI-1 antagonist are well known to the person skilled in the art. In a preferred embodiment, the antagonist specifically binds to PAI-1 (protein or nucleic sequence (DNA or mRNA)) in a sufficient manner to inhibit the biological activity of PAI-1. Binding to PAI-1 and inhibition of the biological activity of PAI-1 may be determined by any competing assays well known in the art. For example, the assay may consist in determining the ability of the agent to be tested as a PAI-1 antagonist to bind to PAI-1. The binding ability is reflected by the Kd measurement. The term “Kd”, as used herein, is intended to refer to the dissociation constant, which is obtained from the ratio of Kd to Ka (i.e. Kd/Ka) and is expressed as a molar concentration (M). Kd values for binding biomolecules can be determined using methods well established in the art. In specific embodiments, an antagonist that “specifically binds to PAI-1” is intended to refer to an inhibitor that binds to human PAI-1 polypeptide with a Kd of 1 µM or less, 100 nM or less, 10 nM or less, or 3 nM or less. Then a competitive assay may be settled to determine the ability of the agent to inhibit biological activity of PAI-1. The functional assays based on general fear conditioning procedures may be envisaged such as evaluating the ability to inhibit processes associated with PTSD-like memories through restoration of tPA activity which mediated proteolytic processing of pro-BDNF to mature BDNF by plasmin (see example 1 and FIGS. 4 to 5) (see also Kaouane et al Science 2012).

The skilled in the art can easily determine whether a PAI-1 antagonist neutralizes, blocks, inhibits, abrogates, reduces or interferes with a biological activity of PAI-1. To check whether the PAI-1 antagonist binds to PAI-1 and/or is able to inhibit processes associated with PTSD-like memories (for instance, through restoration of tPA/plasmin activity) in the same way than the initially characterized inhibitor of PAI-1, binding assay and/or a tPA activity assay may be performed with each antagonist. For instance restoration of tPA activity can be assessed by detecting active tPA with specific antibody (immunoblotting analysis), and/or by assessment of enzymatic activity of tPA on plasmin formation in presence of plasminogen in biological samples. Restoration of tPA activity can also be assessed by detecting mature BDNF and/or P-TrkB and/or P-Erk1/2^{MAPK} with specific antibodies by immunoblotting, ELISA or Alpha technology as described in the Examples 1 section (FIGS. 1, 2 and 3) (see also Tomaselli-Zanese J Neurosci Methods 2020).

Accordingly, the PAI-1 antagonist may be a molecule that binds to PAI-1 selected from the group consisting of small organic molecules antibodies, aptamers, and polypeptides.

The skilled in the art can easily determine whether a PAI-1 antagonist neutralizes, blocks, inhibits, abrogates, reduces or interferes with a biological activity of PAI-1: (i) binding to PAI-1 (protein or nucleic sequence (DNA or mRNA)) and/or (ii), inducing PTSD-like memories through blocking the tPA/plasmin activity.

Accordingly, in a specific embodiment the PAI-1 antagonist directly binds to PAI-1 (protein or nucleic sequence (DNA or mRNA)) and allows to promote tPA/plasmin activity to mediate the proteolytic processing of pro-BDNF to mature BDNF.

Thus in a second aspect the present invention also relates to a PAI-1 antagonist for use in a method to activate the tPA/plasmin activity of a patient affected with pathological conditions associated with intense stress.

The terms “pathological conditions associated with intense stress” refer to or describe the pathological condition that is typically characterized by behavioral disorders associated with intense or chronic stress which can alter memory consolidation. Examples of pathological conditions that are associated with intense stress include Substance Use Disorders (SUD), depressive-like and anxiety-like disorders, in particular Post-Traumatic Stress Disorder (PTSD) (1-4).

Preferably the pathological conditions associated with intense stress is Post-Traumatic Stress Disorder (PTSD).

The term “Post-Traumatic Stress Disorder″ or″ PTSD” refers to or describe a severe stress-related disorder that is developed by 10 to 20% of subjects experiencing a strong traumatic life event (e.g. rapes, terrorist attacks or military fights) (7;8). PTSD is characterized by recurrent and intrusive recollections of the trauma due to the inability of the individual to restrict fear to the appropriate predictor of the threat (5;7;9). In humans, it was shown to be associated with a hippocampal dysfunction that might contribute to the deficit of contextual memory of the trauma (9-11). This progressive shift from adaptive to deleterious consequences as a function of stress intensity follows an inverted-U pattern and has been known since the beginning of the twentieth century (12; 13). The main treatments for subject affected with PTSD are counselling (psychotherapy) and medication. Antidepressants of the selective serotonin reuptake inhibitor type (SSRI) are the first-line medications for PTSD (Berger W, et al (2009). Progress in Neuro-Psychopharmacology & Biological Psychiatry. 33 (2): 169-80). While many medications do not have enough evidence to support their use, three SSRI (fluoxetine, paroxetine, and venlafaxine) have been shown to have a small to modest benefit over placebo (Hoskins M, et al (2015). The British Journal of Psychiatry. 206 (2): 93-100).

Accordingly there is a medical need to specifically treat PTSD patient with new therapeutical approach

Examples of PAI-1 inhibitors include but are not limited to any of the I PAI-1 inhibitors described in Forthenberry Y. Expert Opinion on Therapeutic Patents, 23:7, 801-815 ((2013) all of which are herein incorporated by reference.

Typically, a PAI-1 inhibitors according to the invention includes but is not limited to:
A) Inhibitor of PAI-1 activity such as :
   i. PAI-1 inhibitors small molecule such as: indole oxyacetyl amino acetic acid derivatives such as PAI-039 (Tiplaxtinin) and derived compounds, oxazolo-naphthyl acids such as PAI-749 and derived compounds, Polyphenolic inhibitors such as tannic acid, epigallocatechin-3,5-digallate (EGCDG), epigallocatechin gallate (EGCG) gallic acid (naturally occurring polyphenol) and CDE-066 and and derived compounds, dimeric 2-acylamino-3-thiophenecarboxylic acid derivatives such as TM5275 and derived compounds, and IMD-1622 coumpound [3-(3,4-dichlorobenzyl)-5-(3,4,5-trihydroxy-benzylidene)-thiazolidine-2,4-dione
   ii. Anti-PAI-1 antibody such as; Humanized PAI-1 antibodies and antigen-binding fragments derived murine monoclonal antibody (MA-33B8), bispecific inhibitor Db-TCK26D6x33H1 F7 based on monoclonal antibodies targeting PAI-1 and TAFI (Thrombin-Activatable Fibrinolysis Inhibitor)
   iii. Defibrotide sodium salt of a mixture of single-stranded oligodeoxyribonucleotides derived from porcine mucosal DNA
   iv. Polypeptide such as PAI-1 mutant or enzyme (protease or peptidase) that are capable of inhibiting PAI-1 activity
B) Inhibitor of PAI-1 gene expression selected from the list consisting of antisense oligonucleotide, nuclease, siRNA, shRNA or ribozyme nucleic acid sequence.

### A) Inhibitor of PAI-1 Activity

### Small Organic Molecule

In one embodiment, the PAI-1 antagonist is a small organic molecule. As used herein, the term “small organic molecule” refers to a molecule of size comparable to those organic molecules generally used in pharmaceuticals. The term excludes biological macromolecules (e.g.; proteins, nucleic acids, etc.); preferred small organic molecules range in size up to 2000 Da, and most preferably up to about 1000 Da.

Several PAI-1 inhibitors are disclosed below.

In a particular embodiment, the PAI-1 antagonist according to the invention is a small organic molecule such as:
i. Indole oxyacetyl amino acetic acid derivatives such as PAI-039 (also known as Tiplaxtinin / Cas Number 393105-53-8) and derived compounds (described in Elokdah H, et al J Med Chem. 2004 Jul 1; 47(14):3491-4.) PAI-039 (1H-indole-3-acetic acid, a-oxo-1-(phenylmethyl)-5-(4-trifluoro methoxy phenyl) is the most characterized PAI-1 inhibitor as many of the subsequent inhibitors are based on improving the potency of PAI-039.
   The Agent is disclosed in International Patent Application WO2004052893, WO2004052856). PAI-039 has the following structure: 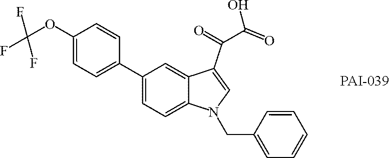 [SMILES: O=C(O)C(=O)c1cn(Cc2ccccc2)c2ccc(-c3ccc(OC(F)(F)F)cc3)cc12]
ii. Oxazolo-naphthyl acids such as PAI-749 and derived compounds. PAI-749 (1-benzyl-3-pentyl-2[-(1H-tetrazol-5-ylmethoxy)naphthalene-2-yl]-1Hindole) is a potent and selective synthetic antagonist of PAI-1, was shown to neutralize PAI-1 activity by means of a dual mechanism of action (Gardell SJ, et al. Mol Pharmacol 2007;72:897-906). It inhibited the activity of PAI-1 both by preventing it from interacting with tPA and by rendering it susceptible to plasmin mediated proteolytic degradation (Gardell SJ, et al. Mol Pharmacol 2007;72:897-906). This PAI-749 (and derived compounds) is disclosed in International Patent Application WO2006023865, WO200602866). PAI-749 has the following structure:

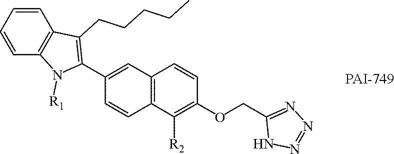

iii. Polyphenolic PAI-1 inhibitors (described in Cale JM, et al. J Biol Chem 2010;285:7892-902 and Skrzypczak-et al Int J Mol Med 2010;26:45-50 ) such as tannic acid, epigallocatechin-3,5-digallate (EGCDG), epigallocatechin gallate (EGCG) gallic acid (naturally occurring polyphenol) and CDE-066 and derived compounds (Synthetic polyphenol). These Polyphenolic PAI-1inhibitors have IC50 (half maximal inhibitory concentration) values ranging from 10 to 200 nM, rendering them very potent inhibitors. The tests showed that each compound disrupts the interaction of PAI-1 with its target protease in the presence of VN. In addition, it appears that they are reversible. Of the compounds tested, two showed efficacy in ex vivo plasma and one blocked PAI-1 activity in vivo in PAI-1-overexpressing mice (Cale JM, et al. JBC 2010). Unlike PAI-039, one of the synthetic inhibitors tested, CDE-066 effectively inhibits PAI-1 in the presence of VN.
   These compounds (naturally occurring and synthesized) are disclosed in International Patent Application WO2008131047). They have the following structure: 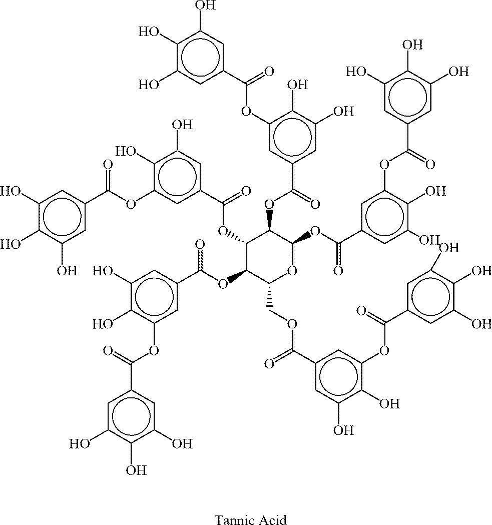 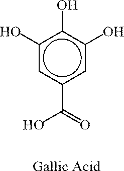 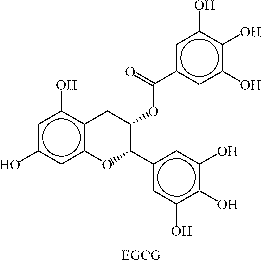 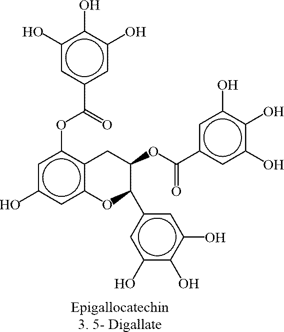
iv Carboxylic acid derivatives PAI inhibitors such as TM5007 and TM5275 and derived compounds. A screening technique (involving virtual screening by docking simulations) identified this molecule. Despite the potency of TM5007 as PAI-1 inhibitors in vitro, their pharmacokinetic profile was not ideal as oral therapeutic molecules. TM5275, display improved oral pharmacokinetic properties. Its efficacy was successfully tested in rats and nonhuman primates, such that TM5275 displayed an effective antithrombotic ability. Even more recently, research has shown that TM5275 blocks TGF-B1-induced lung fibrosis. Hence, it is possible that TM5275 has therapeutic potential in fibrotic lung disease (Huang WT, et al.. Am J Respir Cell Mol Biol 2012;46:87-95)
   This TM5275 (and derived compounds) is disclosed in International Patent Application WO201013022 and has the following structure: 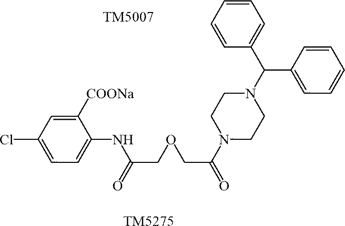
v. Another carboxylic acid derivatives PAIinhibitors such as IMD-1622 compound [3-(3,4-dichlorobenzyl)-5-(3,4,5-trihydroxy-benzylidene)-thiazolidine-2,4-dione) was designed by a virtual screening technique conducted by researchers at the Institute of Medical and Molecular Design (Tokyo, Japan). Suzuki et al. demonstrated the chemical’s ability to inhibit the activity of both mouse and rat PAI-1. Using a rat aorta--vein shunt model, they showed that IMD-1622 decreased thrombus weight (compared to control) and suppressed intimal thickening [Suzuki J,, et al. Expert Opin Ther Targets 2008;12:783-94). Subsequent studies by these researchers showed that IMD-1622 suppresses rat autoimmune myocarditis [Suzuki J, et al. Expert Opin Ther Targets 2008;12:1313-20)].
   IMD-1622 compound (and derived compound) is disclosed in International Patent Application WO2009125606 and has the following structure:

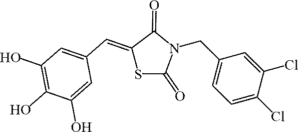 [SMILES: O=C1S/C(=C\c2cc(O)c(O)c(O)c2)C(=O)N1Cc1ccc(Cl)c(Cl)c1]

### Antibody

In another embodiment, the PAI-1 antagonist is an antibody (the term including antibody fragment or portion) that can block directly or indirectly the interaction of PAI-1 with tPA or uPA or with uPA/uPAR complex.

In preferred embodiment, the PAI-1 antagonist may consist in an antibody directed against the PAI-1, in such a way that said antibody impairs the binding of a PAI-1 to tPA or uPA (or uPA/uPAR) and able of neutralizing, blocking, inhibiting, abrogating, reducing or interfering with the biological activities of PAI-1 (“neutralizing antibody”).

Then, for this invention, neutralizing antibody of PAI-1 are selected as above described for their capacity to (i) bind to PAI-1 (protein) and/or (ii) and allow to promote tPA/plasmin activity to mediate the proteolytic processing of pro-BDNF to mature BDNF.

In one embodiment of the antibodies or portions thereof described herein, the antibody is a monoclonal antibody. In one embodiment of the antibodies or portions thereof described herein, the antibody is a polyclonal antibody. In one embodiment of the antibodies or portions thereof described herein, the antibody is a humanized antibody. In one embodiment of the antibodies or portions thereof described herein, the antibody is a chimeric antibody. In one embodiment of the antibodies or portions thereof described herein, the portion of the antibody comprises a light chain of the antibody. In one embodiment of the antibodies or portions thereof described herein, the portion of the antibody comprises a heavy chain of the antibody. In one embodiment of the antibodies or portions thereof described herein, the portion of the antibody comprises a Fab portion of the antibody. In one embodiment of the antibodies or portions thereof described herein, the portion of the antibody comprises a F(ab′)2 portion of the antibody. In one embodiment of the antibodies or portions thereof described herein, the portion of the antibody comprises a Fc portion of the antibody. In one embodiment of the antibodies or portions thereof described herein, the portion of the antibody comprises a Fv portion of the antibody. In one embodiment of the antibodies or portions thereof described herein, the portion of the antibody comprises a variable domain of the antibody. In one embodiment of the antibodies or portions thereof described herein, the portion of the antibody comprises one or more CDR domains of the antibody.

As used herein, “antibody” includes both naturally occurring and non-naturally occurring antibodies. Specifically, “antibody” includes polyclonal and monoclonal antibodies, and monovalent and divalent fragments thereof. Furthermore, “antibody” includes chimeric antibodies, wholly synthetic antibodies, single chain antibodies, and fragments thereof. The antibody may be a human or nonhuman antibody. A nonhuman antibody may be humanized by recombinant methods to reduce its immunogenicity in man.

Antibodies are prepared according to conventional methodology. Monoclonal antibodies may be generated using the method of Kohler and Milstein (Nature, 256:495, 1975). To prepare monoclonal antibodies useful in the invention, a mouse or other appropriate host animal is immunized at suitable intervals (e.g., twice-weekly, weekly, twice-monthly or monthly) with antigenic forms of PAI-1. The animal may be administered a final “boost” of antigen within one week of sacrifice. It is often desirable to use an immunologic adjuvant during immunization. Suitable immunologic adjuvants include Freund’s complete adjuvant, Freund’s incomplete adjuvant, alum, Ribi adjuvant, Hunter’s Titermax, saponin adjuvants such as QS21 or Quil A, or CpG-containing immunostimulatory oligonucleotides. Other suitable adjuvants are well-known in the field. The animals may be immunized by subcutaneous, intraperitoneal, intramuscular, intravenous, intranasal or other routes. A given animal may be immunized with multiple forms of the antigen by multiple routes.

Briefly, the recombinant PAI-1 may be provided by expression with recombinant cell lines or bacteria. Recombinant form of PAI-1 may be provided using any previously described method. Following the immunization regimen, lymphocytes are isolated from the spleen, lymph node or other organ of the animal and fused with a suitable myeloma cell line using an agent such as polyethylene glycol to form a hydridoma. Following fusion, cells are placed in media permissive for growth of hybridomas but not the fusion partners using standard methods, as described (Coding, Monoclonal Antibodies: Principles and Practice: Production and Application of Monoclonal Antibodies in Cell Biology, Biochemistry and Immunology, 3rd edition, Academic Press, New York, 1996). Following culture of the hybridomas, cell supernatants are analyzed for the presence of antibodies of the desired specificity, i.e., that selectively bind the antigen. Suitable analytical techniques include ELISA, flow cytometry, immunoprecipitation, and western blotting. Other screening techniques are well-known in the field. Preferred techniques are those that confirm binding of antibodies to conformationally intact, natively folded antigen, such as non-denaturing ELISA, flow cytometry, and immunoprecipitation.

Significantly, as it is well-known in the art, only a small portion of an antibody molecule, the paratope, is involved in the binding of the antibody to its epitope (see, in general, Clark, W. R. (1986) The Experimental Foundations of Modern Immunology Wiley & Sons, Inc., New York; Roitt, I. (1991) Essential Immunology, 7th Ed., Blackwell Scientific Publications, Oxford). The Fc′ and Fc regions, for example, are effectors of the complement cascade but are not involved in antigen binding. An antibody from which the pFc′ region has been enzymatically cleaved, or which has been produced without the pFc′ region, designated an F(ab′)2 fragment, retains both of the antigen binding sites of an intact antibody. Similarly, an antibody from which the Fc region has been enzymatically cleaved, or which has been produced without the Fc region, designated an Fab fragment, retains one of the antigen binding sites of an intact antibody molecule. Proceeding further, Fab fragments consist of a covalently bound antibody light chain and a portion of the antibody heavy chain denoted Fd. The Fd fragments are the major determinant of antibody specificity (a single Fd fragment may be associated with up to ten different light chains without altering antibody specificity) and Fd fragments retain epitope-binding ability in isolation.

Within the antigen-binding portion of an antibody, as is well-known in the art, there are complementarity determining regions (CDRs), which directly interact with the epitope of the antigen, and framework regions (FRs), which maintain the tertiary structure of the paratope (see, in general, Clark, 1986; Roitt, 1991). In both the heavy chain Fd fragment and the light chain of IgG immunoglobulins, there are four framework regions (FR1 through FR4) separated respectively by three complementarity determining regions (CDR1 through CDRS). The CDRs, and in particular the CDRS regions, and more particularly the heavy chain CDRS, are largely responsible for antibody specificity.

It is now well-established in the art that the non CDR regions of a mammalian antibody may be replaced with similar regions of conspecific or heterospecific antibodies while retaining the epitopic specificity of the original antibody. This is most clearly manifested in the development and use of “humanized” antibodies in which non-human CDRs are covalently joined to human FR and/or Fc/pFc′ regions to produce a functional antibody.

This invention provides in certain embodiments compositions and methods that include humanized forms of antibodies. As used herein, “humanized” describes antibodies wherein some, most or all of the amino acids outside the CDR regions are replaced with corresponding amino acids derived from human immunoglobulin molecules. Methods of humanization include, but are not limited to, those described in U.S. Pat. Nos. 4,816,567,5,225,539,5,585,089, 5,693,761, 5,693,762 and 5,859,205, which are hereby incorporated by reference. The above U.S. Pat. Nos. 5,585,089 and 5,693,761, and WO 90/07861 also propose four possible criteria which may be used in designing the humanized antibodies. The first proposal was that for an acceptor, use a framework from a particular human immunoglobulin that is unusually homologous to the donor immunoglobulin to be humanized, or use a consensus framework from many human antibodies. The second proposal was that if an amino acid in the framework of the human immunoglobulin is unusual and the donor amino acid at that position is typical for human sequences, then the donor amino acid rather than the acceptor may be selected. The third proposal was that in the positions immediately adjacent to the 3 CDRs in the humanized immunoglobulin chain, the donor amino acid rather than the acceptor amino acid may be selected. The fourth proposal was to use the donor amino acid reside at the framework positions at which the amino acid is predicted to have a side chain atom within 3A of the CDRs in a three dimensional model of the antibody and is predicted to be capable of interacting with the CDRs. The above methods are merely illustrative of some of the methods that one skilled in the art could employ to make humanized antibodies. One of ordinary skill in the art will be familiar with other methods for antibody humanization.

In one embodiment of the humanized forms of the antibodies, some, most or all of the amino acids outside the CDR regions have been replaced with amino acids from human immunoglobulin molecules but where some, most or all amino acids within one or more CDR regions are unchanged. Small additions, deletions, insertions, substitutions or modifications of amino acids are permissible as long as they would not abrogate the ability of the antibody to bind a given antigen. Suitable human immunoglobulin molecules would include IgG1, IgG2, IgG3, IgG4, IgA and IgM molecules. A “humanized” antibody retains a similar antigenic specificity as the original antibody. However, using certain methods of humanization, the affinity and/or specificity of binding of the antibody may be increased using methods of “directed evolution”, as described by Wu et al., J. Mol. Biol. 294:151, 1999, the contents of which are incorporated herein by reference.

Fully human monoclonal antibodies also can be prepared by immunizing mice transgenic for large portions of human immunoglobulin heavy and light chain loci. See, e.g., U.S. Pat. Nos. 5,591,669, 5,598,369, 5,545,806, 5,545,807, 6,150,584, and references cited therein, the contents of which are incorporated herein by reference. These animals have been genetically modified such that there is a functional deletion in the production of endogenous (e.g., murine) antibodies. The animals are further modified to contain all or a portion of the human germ-line immunoglobulin gene locus such that immunization of these animals will result in the production of fully human antibodies to the antigen of interest. Following immunization of these mice (e.g., XenoMouse (Abgenix), HuMAb mice (Medarex/GenPharm)), monoclonal antibodies can be prepared according to standard hybridoma technology. These monoclonal antibodies will have human immunoglobulin amino acid sequences and therefore will not provoke human anti-mouse antibody (KAMA) responses when administered to humans.

In vitro methods also exist for producing human antibodies. These include phage display technology (U.S. Pat. Nos. 5,565,332 and 5,573,905) and in vitro stimulation of human B cells (U.S. Pat. Nos. 5,229,275 and 5,567,610). The contents of these patents are incorporated herein by reference.

As the PAI-1 in the context of the present invention is target located in the brain region (hippocampus), the antibody of the invention acting as an activity inhibitor could be an antibody fragment without Fc fragment.

Thus, as will be apparent to one of ordinary skill in the art, the present invention also provides for F(ab′) 2 Fab, Fv and Fd fragments; chimeric antibodies in which the Fc and/or FR and/or CDR1 and/or CDR2 and/or light chain CDR3 regions have been replaced by homologous human or non-human sequences; chimeric F(ab′)2 fragment antibodies in which the FR and/or CDR1 and/or CDR2 and/or light chain CDR3 regions have been replaced by homologous human or non-human sequences; chimeric Fab fragment antibodies in which the FR and/or CDR1 and/or CDR2 and/or light chain CDR3 regions have been replaced by homologous human or non-human sequences; and chimeric Fd fragment antibodies in which the FR and/or CDR1 and/or CDR2 regions have been replaced by homologous human or non-human sequences. The present invention also includes so-called single chain antibodies.

The various antibody molecules and fragments may derive from any of the commonly known immunoglobulin classes, including but not limited to IgA, secretory IgA, IgE, IgG and IgM. IgG subclasses are also well known to those in the art and include but are not limited to human IgGl, IgG2, IgG3 and IgG4.

In another embodiment, the antibody according to the invention is a single domain antibody. The term “single domain antibody” (sdAb) or “VHH” refers to the single heavy chain variable domain of antibodies of the type that can be found in Camelid mammals which are naturally devoid of light chains. Such VHH are also called “nanobody®”. According to the invention, sdAb can particularly be llama sdAb.

The skilled artisan can use routine technologies to use the antigen-binding sequences of these antibodies (e.g., the CDRs) and generate humanized antibodies for treatment of pathological conditions associated with intense stress (such as Post-Traumatic Stress Disorder (PTSD) as disclosed herein.

Several monoclonal antibodies to PAI-1 have been characterized and shown to inhibit PAI-1 activity (see Gils A, et al. CurrMed Chem 2004;11:2323-34).

The skilled artisan can use routine technologies to use the antigen-binding sequences of these antibodies (e.g., the CDRs) and generate humanized antibodies for treatment of pathological conditions associated with intense stress (such as Post-Traumatic Stress Disorder (PTSD) as disclosed herein.

Examples of monoclonal or humanized antibodies that can be used according to the invention include:
i) monoclonal antibodies from Abbot Laboratories (described in WO2011139974A2, US2012114652) The monoclonal antibodies bind to PAI-1 with an affinity ranging from 5 to 200 pM. It is claimed that they bind to PAI-1 in complex with VN. Their binding is specific to PAI-1, has no binding to either PAI-2 or PAI-3 are described. The antibodies work by disrupting the interaction of PAI-1 with tPA.
ii) Humanized PAI-1 antibodies and antigen-binding fragments from Cisthera, Inc (described in WO200933095). They were genetically engineered using a previously characterized murine monoclonal antibody (MA-33B8) (Verhamme I, et al. J Biol Chem 1999;274:17511-17). The humanized antibodies bind to PAI-1 causing it to convert to its latent conformation while also increasing its cleavage. Consequently, it was shown to decrease PAI-1 by interacting with plasminogen activators (tPA and uPA). The antibodies cross-react with other species, including mouse, rabbit, rat, and human PAI-1. Additionally, these antibodies can be used to detect and capture agents during purification studies
iii) The bispecific inhibitor Db-TCK26D6x33H1 F7 based on monoclonal antibodies targeting PAI-1 and TAFI (Thrombin-Activatable Fibrinolysis Inhibitor) and (described in WO2015118147) This bispecific antibody is based on the successful generation of stable scFvs with preserved inhibitory capacity of the parental antibodies (MA-TCK26D6 and MA-33H1 F7) which respectively target and inhibit TAFI and PAI-1. This bispecific inhibitor is destinated to be used for treating thrombotic disorders, such as acute thrombotic disorders like stroke and thromboembolism. This bispecific antibody shows efficacy in the presence or the absence of tPA.

### Aptamer

In another embodiment, the PAI-1 antagonist is an aptamer directed against PAI-1. Aptamers are a class of molecule that represents an alternative to antibodies in term of molecular recognition. Aptamers are oligonucleotide or oligopeptide sequences with the capacity to recognize virtually any class of target molecules with high affinity and specificity. Such ligands may be isolated through Systematic Evolution of Ligands by EXponential enrichment (SELEX) of a random sequence library, as described in Tuerk C. and Gold L., 1990. The random sequence library is obtainable by combinatorial chemical synthesis of DNA. In this library, each member is a linear oligomer, eventually chemically modified, of a unique sequence. Possible modifications, uses and advantages of this class of molecules have been reviewed in Jayasena S.D., 1999. Peptide aptamers consists of a conformationally constrained antibody variable region displayed by a platform protein, such as E. coli Thioredoxin A that are selected from combinatorial libraries by two hybrid methods (Colas et al., 1996).

Then, for this invention, neutralizing aptamers of PAI-1 are selected as above described for their capacity to (i) bind to PAI-1 and/or (ii) and allow to promote tPA/plasmin activity to mediate the proteolytic processing of pro-BDNF to mature BDNF.

Examples of neutralizing RNA aptamers of PAI-1 that can be used according to the invention are disclosed in Blake CM, et al Oligonucleotides 2009;19:117-28 and Madsen JB, et al. RNA Biochemistry 2010;49:4103-15.

### Defibrotide Sodium

Defibrotide, sold under the brandname Defitelio, is a mixture of single-stranded oligonucleotides that is derived from porcine mucosal DNA purified from the intestinal mucosa of pigs. It is used to treat veno-occlusive disease of the liver of people having a bone marrow transplant. (Defibrotide is a polydisperse oligonucleotide with local antithrombotic, anti-ischemic, and anti-inflammatory activity. It binds to the vascular endothelium, modulates platelet activity, promotes fibrinolysis, decreases thrombin generation and activity, and reduces circulating levels of plasminogen activator inhibitor type 1 (PAI-1) increasing tPA function (Richardson MG et al.,Biol Blood Marrow Transplant 16: 1005-1017 (2010) and “Defibrotide sodium label” FDA. March 2016” www.accessdata.fda.gov/drugsatfda_docs/label/2016/208114Orig1s000Lbl.pdf).

### Polypeptide

In another embodiment, the PAI-1 antagonist can be an polypeptide

As used herein, the term “PAI-1 polypeptide antagonist” refers to a polypeptide that specifically binds to PAI-I and be capable of inhibiting PAI-1 biological activity

In a specific embodiments the PAI-1 polypeptide antagonist is as PAI-1 mutant or enzyme (protease or peptidase)

In preferred embodiment, the PAI-1 polypeptide antagonist may consist in a polypeptide directed against the PAI-1 protein, and able of neutralizing, blocking, inhibiting, abrogating, reducing or interfering with the biological activities of PAI-1 (“neutralizing polypeptide ”).

Then, for this invention, neutralizing polypeptide of PAI-1 are selected as above described for their capacity to (i) bind to PAI-1 (protein) and/or (ii) and to allow to promote tPA/plasmin activity to mediate the proteolytic processing of pro-BDNF to mature BDNF.

Examples of PAI-1 polypeptide antagonist that can be used according to the invention include:
i) The enzyme (protease or peptidase) inhibitor of PAI-1 activity (described in WO200747205). The mechanism by which these enzymes inhibit PAI-1 involves binding to a2-macroglobin in such a way that it inhibits protease interaction with protein substrates. Several enzymes such as Seaprose Nattokinase Bromelain Papain and Serapeptaare, are effective at inhibiting PAI-1 with IC50 values ranging from 0.6 to 22.8 µg Seaprose, when given orally to patients with high PAI-1 levels for two weeks, showed decreases in PAI-1 activity to within normal ranges. Effect on plasmin, D-dimer, and LDL levels, all of which fell to normal during treatment, but returned to high levels when the treatment stopped. Hence, the Seaprose inhibits PAI-1 activity in patient and results in improved cardiovascular health. Hence, these enzymes inhibit PAI-1 activity (in vivo, in vitro, and clinical).
ii) The polypeptides ligands and/or modulators of PAI-1 activity (described in WO200747205). Vectors containing polypeptide/polynucleotide sequences that are transfected into cells, alter PAI-1 activity. These polypeptides ligands/modulators can be used to treat or prevent atherosclerosis and/or fibrosis.
iii) The specific PAI-1 antagonist PAItrap derived from inactivated urokinase (uPA Mutant) described in Gong L. et al J Cell Mol Med . 2016 Oct;20(10):1851-60. PAItrap is the serine protease domain of urokinase containing active-site mutation (S195A) and four additional mutations (G37bR-R217L-C122AN145Q). PAItrap inhibits human recombinant PAI-1 with high potency (Kd = 0.15 nM) and high specificity. In vitro using human plasma, PAItrap showed significant thrombolytic activity by inhibiting endogenous PAI-1. In vivo, PAItrap reduced fibrin generation and inhibited platelet accumulation following vascular injury.

### B) Inhibitor of PAI-1 Gene Expression

In still another embodiment, the PAI-1 antagonist is an inhibitor of PAI-1 gene expression. An “inhibitor of expression” refers to a natural or synthetic compound that has a biological effect to inhibit the expression of a gene. Therefore, an “inhibitor of PAI-1 gene expression” denotes a natural or synthetic compound that has a biological effect to inhibit the expression of PAI-1 gene.

In a preferred embodiment of the invention, said inhibitor of PAI-1 gene expression is antisense oligonucleotide, nuclease, siRNA, shRNA or ribozyme nucleic acid sequence.

Inhibitors of PAI-1 gene expression for use in the present invention may be based on antisense oligonucleotide constructs. Antisense oligonucleotides, including antisense RNA molecules and antisense DNA molecules, would act to directly block the translation of PAI-1 mRNA by binding thereto and thus preventing protein translation or increasing mRNA degradation, thus decreasing the level of PAI-1, and thus activity, in a cell. For example, antisense oligonucleotides of at least about 15 bases and complementary to unique regions of the mRNA transcript sequence encoding PAI-1 can be synthesized, e.g., by conventional phosphodiester techniques and administered by e.g., intravenous injection or infusion. Methods for using antisense techniques for specifically inhibiting gene expression of genes whose sequence is known are well known in the art (e.g. see U.S. Pat. Nos. 6,566,135; 6,566,131; 6,365,354; 6,410,323; 6,107,091; 6,046,321; and 5,981,732).

Small inhibitory RNAs (siRNAs) can also function as inhibitors of PAI-1 gene expression for use in the present invention. PAI-1 gene expression can be reduced by using small double stranded RNA (dsRNA), or a vector or construct causing the production of a small double stranded RNA, such that PAI-1 gene expression is specifically inhibited (i.e. RNA interference or RNAi). Methods for selecting an appropriate dsRNA or dsRNA-encoding vector are well known in the art for genes whose sequence is known (e.g. see Tuschi, T. et al. (1999); Elbashir, S. M. et al. (2001); Hannon, GJ. (2002); McManus, MT. et al. (2002); Brummelkamp, TR. et al. (2002); U.S. Pat. Nos. 6,573,099 and 6,506,559; and International Patent Publication Nos. WO 01/36646, WO 99/32619, and WO 01/68836).

Example of commercial siRNAs against PAI-1 are available.

Inhibitors of PAI-1 gene expression for use in the present invention may be based nuclease therapy (like Talen or Crispr).

The term “nuclease” or “endonuclease” means synthetic nucleases consisting of a DNA binding site, a linker, and a cleavage module derived from a restriction endonuclease which are used for gene targeting efforts. The synthetic nucleases according to the invention exhibit increased preference and specificity to bipartite or tripartite DNA target sites comprising DNA binding (i.e. TALEN or CRISPR recognition site(s)) and restriction endonuclease target site while cleaving at off-target sites comprising only the restriction endonuclease target site is prevented.

The guide RNA (gRNA) sequences direct the nuclease (i.e. Cas9 protein) to induce a site-specific double strand break (DSB) in the genomic DNA in the target sequence.

Restriction endonucleases (also called restriction enzymes) as referred to herein in accordance with the present invention are capable of recognizing and cleaving a DNA molecule at a specific DNA cleavage site between predefined nucleotides. In contrast, some endonucleases such as for example Fokl comprise a cleavage domain that cleaves the DNA unspecifically at a certain position regardless of the nucleotides present at this position. Therefore, preferably the specific DNA cleavage site and the DNA recognition site of the restriction endonuclease are identical. Moreover, also preferably the cleavage domain of the chimeric nuclease is derived from a restriction endonuclease with reduced DNA binding and/or reduced catalytic activity when compared to the wildtype restriction endonuclease.

According to the knowledge that restriction endonucleases, particularly type II restriction endonucleases, bind as a homodimer to DNA regularly, the chimeric nucleases as referred to herein may be related to homodimerization of two restriction endonuclease subunits. Preferably, in accordance with the present invention the cleavage modules referred to herein have a reduced capability of forming homodimers in the absence of the DNA recognition site, thereby preventing unspecific DNA binding. Therefore, a functional homodimer is only formed upon recruitment of chimeric nucleases monomers to the specific DNA recognition sites. Preferably, the restriction endonuclease from which the cleavage module of the chimeric nuclease is derived is a type llP restriction endonuclease. The preferably palindromic DNA recognition sites of these restriction endonucleases consist of at least four or up to eight contiguous nucleotides. Preferably, the type llP restriction endonucleases cleave the DNA within the recognition site which occurs rather frequently in the genome, or immediately adjacent thereto, and have no or a reduced star activity. The type llP restriction endonucleases as referred to herein are preferably selected from the group consisting of: Pvull, EcoRV, BamHl, Bcnl, BfaSORF1835P, BfiI, Bgll, Bglll, BpuJl, Bse6341, BsoBl, BspD6I, BstYl, Cfr101, Ecl18kl, EcoO1091, EcoRl, EcoRll, EcoRV, EcoR1241, EcoR124ll, HinP11, Hincll, Hindlll, Hpy991, Hpy1881, Mspl, Munl, Mval, Nael, NgoMIV, Notl, OkrAl, Pabl, Pacl, PspGl, Sau3Al, Sdal, Sfil, SgrAl, Thal, VvuYORF266P, Ddel, Eco571, Haelll, Hhall, Hindll, and Ndel.

Example of commercial gRNAs against PAI-1 include, but are not limited to: Human PAI-1 CRISPR gRNA + Cas9 in Lenti Particles (ABIN5231258) from Genomics oneline, PAI-1 CRISPR Plasmids (human) gene knockout, with PAI-1-specific 20 nt guide RNA sequences from Santa Cruz Biotechnology.

Other nuclease for use in the present invention are disclosed in WO 2010/079430, WO2011072246, WO2013045480, Mussolino C, et al (Curr Opin Biotechnol. 2012 Oct;23(5):644-50) and Papaioannou I. et al (Expert Opinion on Biological Therapy, March 2012, Vol. 12, No. 3 : 329-342) all of which are herein incorporated by reference.

Ribozymes can also function as inhibitors of PAI-1 gene expression for use in the present invention. Ribozymes are enzymatic RNA molecules capable of catalyzing the specific cleavage of RNA. The mechanism of ribozyme action involves sequence specific hybridization of the ribozyme molecule to complementary target RNA, followed by endonucleolytic cleavage. Engineered hairpin or hammerhead motif ribozyme molecules that specifically and efficiently catalyze endonucleolytic cleavage of PAI-1 mRNA sequences are thereby useful within the scope of the present invention. Specific ribozyme cleavage sites within any potential RNA target are initially identified by scanning the target molecule for ribozyme cleavage sites, which typically include the following sequences, GUA, GUU, and GUC. Once identified, short RNA sequences of between about 15 and 20 ribonucleotides corresponding to the region of the target gene containing the cleavage site can be evaluated for predicted structural features, such as secondary structure, that can render the oligonucleotide sequence unsuitable. The suitability of candidate targets can also be evaluated by testing their accessibility to hybridization with complementary oligonucleotides, using, e.g., ribonuclease protection assays.

Antisense oligonucleotides, siRNAs and ribozymes useful as inhibitors of PAI-1 gene expression can be prepared by known methods. These include techniques for chemical synthesis such as, e.g., by solid phase phosphoramadite chemical synthesis. Alternatively, antisense RNA molecules can be generated by in vitro or in vivo transcription of DNA sequences encoding the RNA molecule. Such DNA sequences can be incorporated into a wide variety of vectors that incorporate suitable RNA polymerase promoters such as the T7 or SP6 polymerase promoters. Various modifications to the oligonucleotides of the invention can be introduced as a means of increasing intracellular stability and half-life. Possible modifications include but are not limited to the addition of flanking sequences of ribonucleotides or deoxyribonucleotides to the 5′ and/or 3′ ends of the molecule, or the use of phosphorothioate or 2′-O-methyl rather than phosphodiesterase linkages within the oligonucleotide backbone.

Antisense oligonucleotides, siRNAs and ribozymes of the invention may be delivered in vivo alone or in association with a vector. In its broadest sense, a “vector” is any vehicle capable of facilitating the transfer of the antisense oligonucleotide, siRNA or ribozyme nucleic acid to the cells and preferably cells expressing PAI-1. Preferably, the vector transports the nucleic acid within cells with reduced degradation relative to the extent of degradation that would result in the absence of the vector. In general, the vectors useful in the invention include, but are not limited to, plasmids, phagemids, viruses, other vehicles derived from viral or bacterial sources that have been manipulated by the insertion or incorporation of the antisense oligonucleotide, siRNA or ribozyme nucleic acid sequences. Viral vectors are a preferred type of vectors and include, but are not limited to nucleic acid sequences from the following viruses: retrovirus, such as moloney murine leukemia virus, harvey murine sarcoma virus, murine mammary tumor virus, and rouse sarcoma virus; adenovirus, adeno-associated virus; SV40-type viruses; polyoma viruses; Epstein-Barr viruses; papilloma viruses; herpes virus; vaccinia virus; polio virus; and RNA virus such as a retrovirus. One can readily employ other vectors not named but known to the art.

Preferred viral vectors are based on non-cytopathic eukaryotic viruses in which non-essential genes have been replaced with the gene of interest. Non-cytopathic viruses include retroviruses (e.g., lentivirus), the life cycle of which involves reverse transcription of genomic viral RNA into DNA with subsequent proviral integration into host cellular DNA. Retroviruses have been approved for human gene therapy trials. Most useful are those retroviruses that are replication-deficient (i.e., capable of directing synthesis of the desired proteins, but incapable of manufacturing an infectious particle). Such genetically altered retroviral expression vectors have general utility for the high-efficiency transduction of genes in vivo. Standard protocols for producing replication-deficient retroviruses (including the steps of incorporation of exogenous genetic material into a plasmid, transfection of a packaging cell line with plasmid, production of recombinant retroviruses by the packaging cell line, collection of viral particles from tissue culture media, and infection of the target cells with viral particles) are provided in KRIEGLER (A Laboratory Manual,” W.H. Freeman C.O., New York, 1990) and in MURRY (“Methods in Molecular Biology,” vol.7, Humana Press, Inc., Cliffton, N.J., 1991).

Preferred viruses for certain applications are the adenoviruses and adeno-associated viruses, which are double-stranded DNA viruses that have already been approved for human use in gene therapy. The adeno-associated virus can be engineered to be replication deficient and is capable of infecting a wide range of cell types and species. It further has advantages such as, heat and lipid solvent stability; high transduction frequencies in cells of diverse lineages, including hematopoietic cells; and lack of superinfection inhibition thus allowing multiple series of transductions. Reportedly, the adeno-associated virus can integrate into human cellular DNA in a site-specific manner, thereby minimizing the possibility of insertional mutagenesis and variability of inserted gene expression characteristic of retroviral infection. In addition, wild-type adeno-associated virus infections have been followed in tissue culture for greater than 100 passages in the absence of selective pressure, implying that the adeno-associated virus genomic integration is a relatively stable event. The adeno-associated virus can also function in an extrachromosomal fashion.

Other vectors include plasmid vectors. Plasmid vectors have been extensively described in the art and are well known to those of skill in the art. See e.g., SANBROOK et al., “Molecular Cloning: A Laboratory Manual,” Second Edition, Cold Spring Harbor Laboratory Press, 1989. In the last few years, plasmid vectors have been used as DNA vaccines for delivering antigen-encoding genes to cells in vivo. They are particularly advantageous for this because they do not have the same safety concerns as with many of the viral vectors. These plasmids, however, having a promoter compatible with the host cell, can express a peptide from a gene operatively encoded within the plasmid. Some commonly used plasmids include pBR322, pUC18, pUC19, pRC/CMV, SV40, and pBlueScript. Other plasmids are well known to those of ordinary skill in the art. Additionally, plasmids may be custom designed using restriction enzymes and ligation reactions to remove and add specific fragments of DNA. Plasmids may be delivered by a variety of parenteral, mucosal and topical routes. For example, the DNA plasmid can be injected by intramuscular, intradermal, subcutaneous, or other routes. It may also be administered by intranasal sprays or drops, rectal suppository and orally. It may also be administered into the epidermis or a mucosal surface using a gene-gun. The plasmids may be given in an aqueous solution, dried onto gold particles or in association with another DNA delivery system including but not limited to liposomes, dendrimers, cochleate and microencapsulation.

In a preferred embodiment, the antisense oligonucleotide, nuclease (i.e. CrispR), siRNA, shRNA or ribozyme nucleic acid sequences are under the control of a heterologous regulatory region, e.g., a heterologous promoter. The promoter may be specific for the neural cells.

### Diagnostic Methods According to the Invention

A second aspect of the invention consists of a method for assessing a subject’s risk of having or developing (or in vitro diagnosis) pathological conditions associated with intense stress, said method comprising the step of measuring the level of PAI-1 protein in a body fluid sample obtained from said subject wherein the level of PAI-1 is positively correlated with the risk of said subject of having a pathological conditions associated with intense stress.

A high level of PAI-1 is predictive of a high risk of having or developing a pathological conditions associated with intense stress.

A low level of PAI-1 is predictive of a low risk of having or developing a pathological conditions associated with intense stress.

In a specific embodiment, the pathological conditions associated with intense stress is selected from the list consisting of Substance Use Disorders (SUD), depressive-like and anxiety-like disorders, in particular Post-Traumatic Stress Disorder (PTSD).

Preferably, the pathological conditions associated with intense stress is Post-Traumatic Stress Disorder (PTSD)

As used herein, the term “body fluid sample” refers to any biological fluid sample obtained of a subject; Non-limiting examples of such body fluid sample samples include, but are not limited to, blood, serum, plasma, urine, saliva, and cerebrospinal fluid (CSF) and aqueous humor.

In a preferred embodiment, body fluid sample is blood sample and/or urinary sample.

Indeed, the inventors have surprisingly demonstrated that PAI-1 blood level, known until now to be a protein associated with fibrinolysis, is also associated with pathological conditions associated with intense stress and this PAI-1 blood level is increased with stress (see example 2 and FIGS. 6).

In one embodiment, the blood sample to be used in the methods according to the invention is a whole blood sample, a serum sample, or a plasma sample. In a preferred embodiment, the blood sample is a serum sample.

In a particular embodiment, methods of the invention including any semi- or quantitative proteomic methods based on specific antigen/antibody interactions such as ELISA, immunoblotting or Alpha screen technology are suitable for assessing a subject’s risk of having or developing pathological conditions associated with intense stress especially Post-Traumatic Stress Disorder (PTSD) at an early stage:

According to the present invention, the term “early stage of PTSD” refers to the stage of the disease with or before the onset of clinical symptoms of PTSD that typically include flashbacks characterized by nightmares where the subject relives and is confrontated to the stressful event, avoidance of stimuli associated to the traumatic event, hyperactivity (irritability, angry outbursts, insomnia, difficulty to be concentrated) and long lasting alterations of mood and cognition

As used herein, the term “subject” denotes a mammal, such as a rodent, a feline, a canine, and a primate. Preferably, a subject according to the invention is a human.

Measuring the level of PAI-1 can be done by measuring the gene expression level of PAI-1 and can be performed by a variety of techniques well known in the art.

Typically, the expression level of a gene may be determined by determining the quantity of mRNA. Methods for determining the quantity of mRNA are well known in the art. For example, the nucleic acid contained in the samples (e.g., cell or tissue prepared from the patient) is first extracted according to standard methods, for example using lytic enzymes or chemical solutions or extracted by nucleic-acid-binding resins following the manufacturer’s instructions. The extracted mRNA is then detected by hybridization (e. g., Northern blot analysis, in situ hybridization) and/or amplification (e.g., RT-PCR). Other methods of Amplification include ligase chain reaction (LCR), transcription-mediated amplification (TMA), strand displacement amplification (SDA) and nucleic acid sequence-based amplification (NASBA). Typically, the nucleic acid probes include one or more labels, for example to permit detection of a target nucleic acid molecule using the disclosed probes. In various applications, such as in situ hybridization procedures, a nucleic acid probe includes a label (e.g., a detectable label). A “detectable label” is a molecule or material that can be used to produce a detectable signal that indicates the presence or concentration of the probe (particularly the bound or hybridized probe) in a sample. Thus, a labeled nucleic acid molecule provides an indicator of the presence or concentration of a target nucleic acid sequence (e.g., genomic target nucleic acid sequence) (to which the labeled uniquely specific nucleic acid molecule is bound or hybridized) in a sample. A label associated with one or more nucleic acid molecules (such as a probe generated by the disclosed methods) can be detected either directly or indirectly. A label can be detected by any known or yet to be discovered mechanism including absorption, emission and/ or scattering of a photon (including radio frequency, microwave frequency, infrared frequency, visible frequency and ultra-violet frequency photons). Detectable labels include colored, fluorescent, phosphorescent and luminescent molecules and materials, catalysts (such as enzymes) that convert one substance into another substance to provide a detectable difference (such as by converting a colorless substance into a colored substance or vice versa, or by producing a precipitate or increasing sample turbidity), haptens that can be detected by antibody binding interactions, and paramagnetic and magnetic molecules or materials.

Expression level of a gene may be expressed as absolute expression level or normalized expression level. Typically, expression levels are normalized by correcting the absolute expression level of a gene by comparing its expression to the expression of a gene that is not a relevant for assessing a subject’s risk of having or developing (or in vitro diagnosis) pathological conditions associated with intense stress, such as PTSD.

According to the invention, the level of PAI-1 may also be measured by measuring the protein expression level encoding by said gene and can be performed by a variety of techniques well known in the art.

The level of the PAI-1 may be determined by using standard electrophoretic and immunodiagnostic techniques, including immunoassays such as competition, direct reaction such as immunohistochemistry, or sandwich type assays. Such assays include, but are not limited to, Western blots; agglutination tests; enzyme-labelled and mediated immunoassays, such as ELISAs; biotin/avidin type assays; radioimmunoassays; immunoelectrophoresis; immunoprecipitation, etc. The reactions generally include revealing labels such as fluorescent, chemiluminescent, radioactive, enzymatic labels or dye molecules, or other methods for detecting the formation of a complex between the antigen and the antibody or antibodies reacted therewith.

For example, determination of the PAI-1 level can be performed by a variety of techniques and method any well-known method in the art: RIA kits (DiaSorin; IDS, Diasource) ELISA kits (Thermo Fisher, EHTGFBI, R&D DY2935, IDS (manual) IDS (adapted on open analyzers) Immunochemiluminescent automated methods (DiaSorin Liaison, Roche Elecsys family, IDS iSYS) (Janssen et al., 2012).

In a particular embodiment, the methods of the invention comprise contacting the body fluid sample (ie blood and/or urine sample) with a binding partner.

As used therein, binding partner refers to a molecule capable of selectively interacting with PAI-1.

The binding partner may be generally an antibody that may be polyclonal or monoclonal, preferably monoclonal. Polyclonal antibodies directed against PAI-1 can be raised according to known methods by administering the appropriate antigen or epitope to a host animal selected, e.g., from pigs, cows, horses, rabbits, goats, sheep, and mice, among others. Various adjuvants known in the art can be used to enhance antibody production. Although antibodies useful in practicing the invention can be polyclonal, monoclonal antibodies are preferred. Monoclonal antibodies against PAI-1 can be prepared and isolated using any technique that provides for the production of antibody molecules by continuous cell lines in culture. Techniques for production and isolation include are disclosed above. Antibodies useful in practicing the present invention also include anti-PAI-1 including but not limited to F(ab′)2 fragments, which can be generated by pepsin digestion of an intact antibody molecule, and Fab fragments, which can be generated by reducing the disulfide bridges of the F(ab′)2 fragments. Alternatively, Fab and/or scFv expression libraries can be constructed to allow rapid identification of fragments having the desired specificity to PAI-1. For example, phage display of antibodies may be used. In such a method, single-chain Fv (scFv) or Fab fragments are expressed on the surface of a suitable bacteriophage, e.g., M13. Briefly, spleen cells of a suitable host, e.g., mouse that has been immunized with a protein are removed. The coding regions of the VL and VH chains are obtained from those cells that are producing the desired antibody against the protein. These coding regions are then fused to a terminus of a phage sequence. Once the phage is inserted into a suitable carrier, e. g., bacteria, the phage displays the antibody fragment. Phage display of antibodies may also be provided by combinatorial methods known to those skilled in the art. Antibody fragments displayed by a phage may then be used as part of an immunoassay.

In another embodiment, the binding partner may be an aptamer as described above.

The binding partners of the invention such as antibodies or aptamers, may be labeled with a detectable molecule or substance, such as a fluorescent molecule, a radioactive molecule or any others labels known in the art. Labels are known in the art that generally provide (either directly or indirectly) a signal.

As used herein, the term “labeled”, with regard to the binding partner, is intended to encompass direct labeling of the antibody or aptamer by coupling (i.e., physically linking) a detectable substance, such as a radioactive agent or a fluorophore (e.g. fluorescein isothiocyanate (FITC) or phycoerythrin (PE) or Indocyanine (Cy5)) to the antibody or aptamer, as well as indirect labeling of the probe or antibody by reactivity with a detectable substance. An antibody or aptamer of the invention may be labeled with a radioactive molecule by any method known in the art. For example radioactive molecules include but are not limited radioactive atom for scintigraphic studies such as I123, I124, In111, Re186, Re188.

The aforementioned assays generally involve the bounding of the binding partner (i.e. antibody or aptamer) in a solid support. Solid supports which can be used in the practice of the invention include substrates such as nitrocellulose (e. g., in membrane or microtiter well form); polyvinylchloride (e. g., sheets or microtiter wells); polystyrene latex (e.g., beads or microtiter plates); polyvinylidine fluoride; diazotized paper; nylon membranes; activated beads, magnetically responsive beads, and the like. More particularly, an ELISA method can be used, wherein the wells of a microtiter plate are coated with a set of antibodies against PAI-1 protein. A body fluid sample containing or suspected of containing PAI-1 is then added to the coated wells. After a period of incubation sufficient to allow the formation of binding partner-PAI-1 complexes, the plate(s) can be washed to remove unbound material and a labeled secondary binding molecule added. The secondary binding molecule is allowed to react with any captured sample marker protein, the plate washed and the presence of the secondary binding molecule detected using methods well known in the art.

As the binding partner, the secondary binding molecule may be labeled.

Different immunoassays, such as radioimmunoassay or ELISA, have been described in the art.

Measuring the level of PAI-1 with or without immunoassay-based methods may also include separation of the proteins: centrifugation based on the protein’s molecular weight; electrophoresis based on mass and charge; HPLC based on hydrophobicity; size exclusion chromatography based on size; and solid-phase affinity based on the protein’s affinity for the particular solid-phase that is used. Once separated, PAI-1 may be identified based on the known “separation profile” e. g., retention time, for that protein and measured using standard techniques. Alternatively, the separated proteins may be detected and measured by, for example, a mass spectrometer.

In a preferred embodiment, the method for measuring the level of PAI-1 comprises the step of contacting the body fluid sample (ie blood and/or urine sample) with a binding partner capable of selectively interacting with PAI-1 to allow formation of a binding partner-PAI-1 complex.

In more preferred embodiment, the method according to the invention comprises further the steps of separating any unbound material of the body fluid sample (ie blood and/or urine sample) from the binding partner-PAI-1 complex, contacting the binding partner-PAI-1 complex with a labelled secondary binding molecule, separating any unbound secondary binding molecule from secondary binding molecule-PAI-1 complexes and measuring the level of the secondary binding molecule of the secondary binding molecule-PAI-1 complexes.

Typically, a high or a low level of PAI-1 is intended by comparison to a control reference value.

Thus, accordingly to the invention, the term “a high level of PAI-1” refers to a higher level of PAI-1 than a control reference value.

Thus, accordingly to the invention, the term “a low level of PAI-1” refers to a lower level of PAI-1 than a control reference value.

Accordingly, in a particular embodiment, the diagnostic method of the present invention comprising the step of comparing said level of PAI-1 to a control reference value wherein
A high level of PAI-1 is predictive of a high risk of having or developing a pathological conditions associated with intense stress.
A low level of PAI-1 is predictive of a low risk of having or developing a pathological conditions associated with intense stress.

Said reference control values may be determined in regard to the level of PAI-1 present in body fluid sample (ie blood and/or urine sample) taken from one or more healthy subject or to the PAI-1 distribution in a control population.

In one embodiment, the method according to the present invention comprises the step of comparing said level of PAI-1 to a control reference value wherein a high level of PAI-1 compared to said control reference value is predictive of a high risk of having a pathological conditions associated with intense stress and a low level of PAI-1 compared to said control reference value is predictive of a low risk of having a pathological conditions associated with intense stress.

The control reference value may depend on various parameters such as the method used to measure the level of PAI-1 or the gender of the subject.

Control reference values are easily determinable by the one skilled in the art, by using the same techniques as for determining the level of PAI-1 in body fluid sample (ie blood and/or urine samples) previously collected from the patient under testing.

A “reference value” can be a “threshold value” or a “cut-off value”. Typically, a “threshold value” or “cut-off value” can be determined experimentally, empirically, or theoretically. A threshold value can also be arbitrarily selected based upon the existing experimental and/or clinical conditions, as would be recognized by a person of ordinary skilled in the art. The threshold value has to be determined in order to obtain the optimal sensitivity and specificity according to the function of the test and the benefit/risk balance (clinical consequences of false positive and false negative). Typically, the optimal sensitivity and specificity (and so the threshold value) can be determined using a Receiver Operating Characteristic (ROC) curve based on experimental data (see FIGS. 1). Preferably, the person skilled in the art may compare the level of PAI-1 protein expression (protein or nucleic sequence (mRNA)) of the present invention with a defined threshold value. In one embodiment of the present invention, the threshold value is derived from the PAI-1 protein level (or ratio, or score) determined in a body fluid sample (ie blood and/or urine sample) derived from one or more subjects who are responders (to the method according to the invention). In one embodiment of the present invention, the threshold value may also be derived from PAI-1 protein level (or ratio, or score) determined in a skin sample derived from one or more subjects or who are non-responders. Furthermore, retrospective measurement of the PAI-1 protein level (or ratio, or scores) in properly banked historical subject samples may be used in establishing these threshold values.

For example, after determining the expression level of the PAI-1 protein expression (protein or nucleic sequence (mRNA)) in a group of reference, one can use algorithmic analysis for the statistic treatment of the expression levels determined in samples to be tested, and thus obtain a classification standard having significance for sample classification. The full name of ROC curve is receiver operator characteristic curve, which is also known as receiver operation characteristic curve. It is mainly used for clinical biochemical diagnostic tests. ROC curve is a comprehensive indicator that reflects the continuous variables of true positive rate (sensitivity) and false positive rate (1-specificity). It reveals the relationship between sensitivity and specificity with the image composition method. A series of different cut-off values (thresholds or critical values, boundary values between normal and abnormal results of diagnostic test) are set as continuous variables to calculate a series of sensitivity and specificity values. Then sensitivity is used as the vertical coordinate and specificity is used as the horizontal coordinate to draw a curve. The higher the area under the curve (AUC), the higher the accuracy of diagnosis. On the ROC curve, the point closest to the far upper left of the coordinate diagram is a critical point having both high sensitivity and high specificity values. The AUC value of the ROC curve is between 1.0 and 0.5. When AUC>0.5, the diagnostic result gets better and better as AUC approaches 1. When AUC is between 0.5 and 0.7, the accuracy is low. When AUC is between 0.7 and 0.9, the accuracy is moderate. When AUC is higher than 0.9, the accuracy is high. This algorithmic method is preferably done with a computer. Existing software or systems in the art may be used for the drawing of the ROC curve, such as: MedCalc 9.2.0.1 medical statistical software, SPSS 9.0, ROCPOWER.SAS, DESIGNROC.FOR, MULTIREADER POWER.SAS, CREATE-ROC.SAS, GB STAT VI0.0 (Dynamic Microsystems, Inc. Silver Spring, Md., USA), etc.

In some embodiments, the method of the invention comprises the use of a classification algorithm typically selected from Linear Discriminant Analysis (LDA), Topological Data Analysis (TDA), Neural Networks, Support Vector Machine (SVM) algorithm and Random Forests algorithm (RF). In some embodiments, the method of the invention comprises the step of determining the subject response using a classification algorithm. As used herein, the term “classification algorithm” has its general meaning in the art and refers to classification and regression tree methods and multivariate classification well known in the art such as described in US 8,126,690; WO2008/156617. As used herein, the term “support vector machine (SVM)” is a universal learning machine useful for pattern recognition, whose decision surface is parameterized by a set of support vectors and a set of corresponding weights, refers to a method of not separately processing, but simultaneously processing a plurality of variables. Thus, the support vector machine is useful as a statistical tool for classification. The support vector machine non-linearly maps its n-dimensional input space into a high dimensional feature space, and presents an optimal interface (optimal parting plane) between features. The support vector machine comprises two phases: a training phase and a testing phase. In the training phase, support vectors are produced, while estimation is performed according to a specific rule in the testing phase. In general, SVMs provide a model for use in classifying each of n subjects to two or more disease categories based on one k-dimensional vector (called a k-tuple) of biomarker measurements per subject. An SVM first transforms the k-tuples using a kernel function into a space of equal or higher dimension. The kernel function projects the data into a space where the categories can be better separated using hyperplanes than would be possible in the original data space. To determine the hyperplanes with which to discriminate between categories, a set of support vectors, which lie closest to the boundary between the disease categories, may be chosen. A hyperplane is then selected by known SVM techniques such that the distance between the support vectors and the hyperplane is maximal within the bounds of a cost function that penalizes incorrect predictions. This hyperplane is the one which optimally separates the data in terms of prediction (Vapnik, 1998 Statistical Learning Theory. New York: Wiley). Any new observation is then classified as belonging to any one of the categories of interest, based where the observation lies in relation to the hyperplane. When more than two categories are considered, the process is carried out pairwise for all of the categories and those results combined to create a rule to discriminate between all the categories. As used herein, the term “Random Forests algorithm” or “RF” has its general meaning in the art and refers to classification algorithm such as described in US 8,126,690; WO2008/156617. Random Forest is a decision-tree-based classifier that is constructed using an algorithm originally developed by Leo Breiman (Breiman L, “Random forests,” Machine Learning 2001, 45:5-32). The classifier uses a large number of individual decision trees and decides the class by choosing the mode of the classes as determined by the individual trees. The individual trees are constructed using the following algorithm: (1) Assume that the number of cases in the training set is N, and that the number of variables in the classifier is M; (2) Select the number of input variables that will be used to determine the decision at a node of the tree; this number, m should be much less than M; (3) Choose a training set by choosing N samples from the training set with replacement; (4) For each node of the tree randomly select m of the M variables on which to base the decision at that node; (5) Calculate the best split based on these m variables in the training set. In some embodiments, the score is generated by a computer program.

In some embodiments, the method of the present invention comprises a) quantifying the level of PAI-1 protein expression (protein or nucleic sequence (mRNA)) in the body fluid sample (ie blood and/or urine sample); b) implementing a classification algorithm on data comprising the quantified PAI-1 protein so as to obtain an algorithm output; c) determining the probability that the subject will develop a pathological conditions associated with intense stress from the algorithm output of step b).

The algorithm used with the method of the present invention can be performed by one or more programmable processors executing one or more computer programs to perform functions by operating on input data and generating output. The algorithm can also be performed by, and apparatus can also be implemented as, special purpose logic circuitry, e.g., an FPGA (field programmable gate array) or an ASIC (application-specific integrated circuit). Processors suitable for the execution of a computer program include, by way of example, both general and special purpose microprocessors, and any one or more processors of any kind of digital computer. Generally, a processor will receive instructions and data from a read-only memory or a random access memory or both. The essential elements of a computer are a processor for performing instructions and one or more memory devices for storing instructions and data. Generally, a computer will also include, or be operatively coupled to receive data from or transfer data to, or both, one or more mass storage devices for storing data, e.g., magnetic, magneto-optical disks, or optical disks. However, a computer need not have such devices. Moreover, a computer can be embedded in another device. Computer-readable media suitable for storing computer program instructions and data include all forms of non-volatile memory, media and memory devices, including by way of example semiconductor memory devices, e.g., EPROM, EEPROM, and flash memory devices; magnetic disks, e.g., internal hard disks or removable disks; magneto-optical disks; and CD-ROM and DVD-ROM disks. The processor and the memory can be supplemented by, or incorporated in, special purpose logic circuitry. To provide for interaction with a user, embodiments of the invention can be implemented on a computer having a display device, e.g., in non-limiting examples, a CRT (cathode ray tube) or LCD (liquid crystal display) monitor, for displaying information to the user and a keyboard and a pointing device, e.g., a mouse or a trackball, by which the user can provide input to the computer. Other kinds of devices can be used to provide for interaction with a user as well; for example, feedback provided to the user can be any form of sensory feedback, e.g., visual feedback, auditory feedback, or tactile feedback; and input from the user can be received in any form, including acoustic, speech, or tactile input. Accordingly, in some embodiments, the algorithm can be implemented in a computing system that includes a back-end component, e.g., as a data server, or that includes a middleware component, e.g., an application server, or that includes a front-end component, e.g., a client computer having a graphical user interface or a Web browser through which a user can interact with an implementation of the invention, or any combination of one or more such back-end, middleware, or front-end components. The components of the system can be interconnected by any form or medium of digital data communication, e.g., a communication network. Examples of communication networks include a local area network (“LAN”) and a wide area network (“WAN”), e.g., the Internet. The computing system can include clients and servers. A client and server are generally remote from each other and typically interact through a communication network. The relationship of client and server arises by virtue of computer programs running on the respective computers and having a client-server relationship to each other.

“Risk” in the context of the present invention, relates to the probability that an event will occur over a specific time period, as in the conversion to pathological conditions associated with intense stress (such as Post-Traumatic Stress Disorder (PTSD), and can mean a subject’s “absolute” risk or “relative” risk. Absolute risk can be measured with reference to either actual observation post-measurement for the relevant time cohort, or with reference to index values developed from statistically valid historical cohorts that have been followed for the relevant time period. Relative risk refers to the ratio of absolute risks of a subject compared either to the absolute risks of low risk cohorts or an average population risk, which can vary by how clinical risk factors are assessed. Odds ratios, the proportion of positive events to negative events for a given test result, are also commonly used (odds are according to the formula p/(1-p) where p is the probability of event and (1-p) is the probability of no event) to no conversion. Alternative continuous measures, which may be assessed in the context of the present invention, include time to pathological conditions associated with intense stress (such as Post-Traumatic Stress Disorder (PTSD)) conversion risk reduction ratios.

“Risk evaluation,” or “evaluation of risk” in the context of the present invention encompasses making a prediction of the probability, odds, or likelihood that an event or disease state may occur, the rate of occurrence of the event or conversion from one disease state to another, i.e., from a normal condition to a pathological conditions associated with intense stress (such as Post-Traumatic Stress Disorder (PTSD)) condition or to one at risk of developing a pathological conditions associated with intense stress (such as Post-Traumatic Stress Disorder (PTSD)). Risk evaluation can also comprise prediction of future clinical parameters, traditional laboratory risk factor values, or other indices of pathological conditions associated with intense stress (such as Post-Traumatic Stress Disorder (PTSD)), such as cellular population determination in peripheral tissues, in serum or other fluid, either in absolute or relative terms in reference to a previously measured population. The methods of the present invention may be used to make continuous or categorical measurements of the risk of conversion to pathological conditions associated with intense stress (such as Post-Traumatic Stress Disorder (PTSD), thus diagnosing and defining the risk spectrum of a category of subjects defined as being at risk for a pathological conditions associated with intense stress (such as Post-Traumatic Stress Disorder (PTSD). In the categorical scenario, the invention can be used to discriminate between normal and other subject cohorts at higher risk for pathological conditions associated with intense stress (such as Post-Traumatic Stress Disorder (PTSD). In other embodiments, the present invention may be used so as to help to discriminate those having pathological conditions associated with intense stress (such as Post-Traumatic Stress Disorder (PTSD)) from normal.

The invention also relates to the use of PAI-1 as a body fluid (ie blood and/or urine) biomarker of PTSD, especially at early stage. According the present invention, the term “early stage of PTSD” refers to the stage of the disease with or before the onset of clinical symptoms of PTSD that typically include flashbacks characterized by nightmares where the subject relives and is confrontated to the stressful event, avoidance of stimuli associated to the traumatic event, hyperactivity (irritability, angry outbursts, insomnia, difficulty to be concentrated) and long lasting alterations of mood and cognition

### Monitoring Treatments

Monitoring the influence of agents (e.g., drug compounds) on the level of expression of one or more tissue-specific biological markers of the invention can be applied for monitoring the potency of the treated pathological conditions associated with intense stress of the patient with time. For example, the effectiveness of an agent to affect PAI-1 expression or activity can be monitored during treatments of subjects receiving anti-PTSD treatments for instance.

In a specific embodiment, the pathological conditions associated with intense stress is selected from the list consisting of Substance Use Disorders (SUD), depressive-like and anxiety-like disorders, in particular Post-Traumatic Stress Disorder (PTSD).

Preferably, the pathological conditions associated with intense stress is Post-Traumatic Stress Disorder (PTSD)

Accordingly, another object of the invention also relates to method for monitoring the effect of a therapy for treating pathological conditions associated with intense stress in a subject comprising the step of measuring the level of PAI-1 in a first body fluid sample (ie blood and/or urine sample) obtained from said subject at t1 and measuring the level of PAI-1 in a second body fluid sample (ie blood and/or urine sample) obtained from said subject at t2 wherein when t1 is prior to therapy, t2 is during or following therapy, and when t1 is during therapy, t2 is later during therapy or following therapy, and wherein a decrease in the level of PAI-1 in the second sample as compared to the level of PAI-1 in the first sample is indicative of a positive effect of the therapy on pathological conditions associated with intense stress in the treated subject.

In another embodiment, the present invention provides a method for monitoring the effectiveness of treatment of a subject with an agent (e.g., an agonist, antagonist, peptidomimetic, protein, peptide, nucleic acid, small molecule, or other drug candidate) comprising the steps of (i) obtaining a pre- administration blood sample from a subject prior to administration of the agent; (ii) detecting the PAI-1 body fluid level (ie blood and/or urine level); (iii) obtaining one or more post- administration samples from the subject; (iv) detecting PAI-1 body fluid level (ie blood and/or urine level) in the post-administration samples; (v) comparing PAI-1 level in the pre-administration sample with the level of expression in the post-administration sample or samples; and (vi) altering the administration of the agent to the subject accordingly. For example, increased PAI-1 body fluid level (ie blood and/or urine level) during the course of treatment may indicate ineffective dosage and the desirability of increasing the dosage, or indicative to the necessity to change the treatment. Conversely, decreased PAI-1 body fluid level (ie blood and/or urine level) may indicate efficacious treatment and no need to change dosage.

In a specific embodiment, the therapy for treating pathological conditions associated with intense stress is selected from the group consisting of SSRI treatment and/or a PAI-1 antagonists.

According to the invention, a positive effect of the therapy on pathological conditions associated with intense stress indicates that the therapy reverses alleviates, inhibits the progress of the pathological conditions associated with intense stress, or one or more symptoms of such pathological conditions associated with intense stress. In particular, the positive effect of the therapy consists in reducing the number of pathological memories as observed in Post-Traumatic Stress Disorders (PTSD). Most preferably, the positive effect of the therapy leads to the complete depletion of the pathological memories as observed in Post-Traumatic Stress Disorders (PTSD).

Because repeated collection of biological samples from the patient affected with pathological conditions associated with intense stress are needed for performing the monitoring method described above, then preferred biological samples is body fluid sample (ie blood and/or urine samples) susceptible to contain (i) cells originating from the patient’s tissue, or (ii) specific marker expression products synthesized by cells originating from the patients tissue, including nucleic acids and proteins.

### Method of Preventing or Treating Pathological Conditions Associated With Intense Stress

The present invention further contemplates a method of preventing or treating pathological conditions associated with intense stress in a subject comprising administering to the subject a therapeutically effective amount of a PAI-1 antagonist.

In a specific embodiment, the pathological conditions associated with intense stress is selected from the list consisting of Substance Use Disorders (SUD), depressive-like and anxiety-like disorders, in particular Post-Traumatic Stress Disorder (PTSD).

Preferably, the pathological conditions associated with intense stress is Post-Traumatic Stress Disorder (PTSD)

In one aspect, the present invention provides a method of inhibiting pathological conditions associated with intense stress in a subject comprising administering a therapeutically effective amount of a PAI-1 antagonist.

By a “therapeutically effective amount” of a PAI-1 antagonist as described above is meant a sufficient amount of the antagonist to prevent or treat a pathological conditions associated with intense stress. It will be understood, however, that the total daily usage of the compounds and compositions of the present invention will be decided by the attending physician within the scope of sound medical judgment. The specific therapeutically effective dose level for any particular subject will depend upon a variety of factors including the disorder being treated and the severity of the disorder; activity of the specific compound employed; the specific composition employed, the age, body weight, general health, sex and diet of the subject; the time of administration, route of administration, and rate of excretion of the specific compound employed; the duration of the treatment; drugs used in combination or coincidental with the specific polypeptide employed; and like factors well known in the medical arts. For example, it is well known within the skill of the art to start with doses of the compound at levels lower than those required to achieve the desired therapeutic effect and to gradually increase the dosage until the desired effect is achieved. However, the daily dosage of the products may be varied over a wide range from 0.01 to 1,000 mg per adult per day. Preferably, the compositions contain 0.01, 0.05, 0.1, 0.5, 1.0, 2.5, 5.0, 10.0, 15.0, 25.0, 50.0, 100, 250 and 500 mg of the active ingredient for the symptomatic adjustment of the dosage to the subject to be treated. A medicine typically contains from about 0.01 mg to about 500 mg of the active ingredient, preferably from 1 mg to about 100 mg of the active ingredient. An effective amount of the drug is ordinarily supplied at a dosage level from 0.0002 mg/kg to about 20 mg/kg of body weight per day, especially from about 0.001 mg/kg to 7 mg/kg of body weight per day.

The invention also relates to a method for treating a pathological conditions associated with intense stress in a subject having a high level of PAI-1 in a body fluid sample (ie blood and/or urine sample) with a PAI-1 antagonist.

The invention also relates to PAI-1 antagonist for use in the treatment of a PTSD in a subject having a high level of PAI-1 in a body fluid sample (ie blood and/or urine sample).

The above method and use comprise the step of measuring the level of PAI-1 protein expression (protein or nucleic sequence (DNA or mRNA)) in a body fluid sample (ie blood and/or urine sample) obtained from said subject wherein and compared to a reference control value.

A high level of PAI-1 is predictive of a high risk of having or developing a pathological conditions associated with intense stress (such as Post-Traumatic Stress Disorder (PTSD) and means that PAI-1 antagonist could be used.

Typically, a body fluid sample (ie blood and/or urine sample) is obtained from the subject and the level of PAI-1 is measured in this sample. Indeed, decreasing PAI-1 levels would be particularly beneficial in those patients displaying high levels of PAI-1.

In other words, the invention refers to a PAI-1 antagonist for use in the treatment of a PTSD in a subject comprising the step of i) measuring the level of PAI-1 protein expression in a body fluid sample obtained from said subject, ii) compared the level of PAI-1 protein expression with a reference control value and iii) administering to said subject the therapeutically effective amount of a PAI-1 antagonist.

### Pharmaceutical Compositions of the Invention:

The PAI-1 antagonist/ inhibitor of PAI-1 gene expression as described above may be combined with pharmaceutically acceptable excipients, and optionally sustained-release matrices, such as biodegradable polymers, to form therapeutic compositions.

Accordingly, the present invention relates to a pharmaceutical composition comprising a PAI-1 antagonist according to the invention and a pharmaceutically acceptable carrier.

The present invention also relates to a pharmaceutical composition for use in the prevention or treatment of pathological conditions associated with intense stress (such as Post-Traumatic Stress Disorder (PTSD) comprising a PAI-1 antagonist according to the invention and a pharmaceutically acceptable carrier.

“Pharmaceutically” or “pharmaceutically acceptable” refers to molecular entities and compositions that do not produce an adverse, allergic or other untoward reaction when administered to a mammal, especially a human, as appropriate. A pharmaceutically acceptable carrier or excipient refers to a non-toxic solid, semi-solid or liquid filler, diluent, encapsulating material or formulation auxiliary of any type.

In therapeutic applications, compositions are administered to a patient already suffering from a disease, as described, in an amount sufficient to cure or at least partially stop the symptoms of the disease and its complications. An appropriate dosage of the pharmaceutical composition is readily determined according to any one of several well-established protocols. For example, animal studies (for example on mice or rats) are commonly used to determine the maximal tolerable dose of the bioactive agent per kilogram of weight. In general, at least one of the animal species tested is mammalian. The results from the animal studies can be extrapolated to determine doses for use in other species, such as humans for example. What constitutes an effective dose also depends on the nature and severity of the disease or condition, and on the general state of the patient’s health.

In therapeutic treatments, the antagonist contained in the pharmaceutical composition can be administered in several dosages or as a single dose until a desired response has been achieved. The treatment is typically monitored and repeated dosages can be administered as necessary. Compounds of the invention may be administered according to dosage regimens established whenever inactivation of PAI-1 is required.

The daily dosage of the products may be varied over a wide range from 0.01 to 1,000 mg per adult per day. Preferably, the compositions contain 0.01, 0.05, 0.1, 0.5, 1.0, 2.5, 5.0, 10.0, 15.0, 25.0, 50.0, 100, 250 and 500 mg of the active ingredient for the symptomatic adjustment of the dosage to the patient to be treated. A medicament typically contains from about 0.01 mg to about 500 mg of the active ingredient, preferably from 1 mg to about 100 mg of the active ingredient. An effective amount of the drug is ordinarily supplied at a dosage level from 0.0002 mg/kg to about 20 mg/kg of body weight per day, especially from about 0.001 mg/kg to 10 mg/kg of body weight per day. It will be understood, however, that the specific dose level and frequency of dosage for any particular patient may be varied and will depend upon a variety of factors including the activity of the specific compound employed, the metabolic stability, and length of action of that compound, the age, the body weight, general health, sex, diet, mode and time of administration, rate of excretion, drug combination, the severity of the particular condition, and the host undergoing therapy.

In the pharmaceutical compositions of the present invention for oral, sublingual, subcutaneous, intramuscular, intravenous, transdermal, local or rectal administration, the active principle, alone or in combination with another active principle, can be administered in a unit administration form, as a mixture with conventional pharmaceutical supports, to animals and human beings. Suitable unit administration forms comprise oral-route forms such as tablets, gel capsules, powders, granules and oral suspensions or solutions, sublingual and buccal administration forms, aerosols, implants, subcutaneous, transdermal, topical, intraperitoneal, intramuscular, intravenous, subdermal, transdermal, intrathecal and intranasal administration forms and rectal administration forms.

The appropriate unit forms of administration include forms for oral administration, such as tablets, gelatine capsules, powders, granules and solutions or suspensions to be taken orally, forms for sublingual and buccal administration, aerosols, implants, forms for subcutaneous, intramuscular, intravenous, intranasal or intraocular administration and forms for rectal administration.

In the pharmaceutical compositions of the present invention, the active principle is generally formulated as dosage units containing from 0.5 to 1000 mg, preferably from 1 to 500 mg, more preferably from 2 to 200 mg of said active principle per dosage unit for daily administrations.

When preparing a solid composition in the form of tablets, a wetting agent such as sodium laurylsulfate can be added to the active principle optionally micronized, which is then mixed with a pharmaceutical vehicle such as silica, gelatine, starch, lactose, magnesium stearate, talc, gum arabic or the like. The tablets can be coated with sucrose, with various polymers or other appropriate substances or else they can be treated so as to have a prolonged or delayed activity and so as to release a predetermined amount of active principle continuously.

A preparation in the form of gelatin capsules is obtained by mixing the active principle with a diluent such as a glycol or a glycerol ester and pouring the mixture obtained into soft or hard gelatine capsules.

A preparation in the form of a syrup or elixir can contain the active principle together with a sweetener, which is preferably calorie-free, methyl-paraben and propylparaben as an antiseptic, a flavoring and an appropriate color.

The water-dispersible powders or granules can contain the active principle mixed with dispersants or wetting agents, or suspending agents such as polyvinyl-pyrrolidone, and also with sweeteners or taste correctors.

The active principle can also be formulated as microcapsules or microspheres, optionally with one or more carriers or additives.

Among the prolonged-release forms which are useful in the case of chronic treatments, implants can be used. These can be prepared in the form of an oily suspension or in the form of a suspension of microspheres in an isotonic medium.

### FIGURES:

FIGS. 1. PAI-1 expression is increased by Corticosterone. PAI-1 mRNA, measured by qPCR (a), and protein expressions, measured by western blot (b, c) in response to 100 nM and 1000 nM of Cort for 3 h (180 min) in PC12 cells. PAI-1, tPA, P-TrkB, P-Erk1/2^{MAPK} proteins measured by western blot (d, e) in dorsal hippocampal slices of Sprague-Dawley rats incubated with 10 nM and 1000 nM of Cort for 1 h (60 min) and 3 h (180 min). α-tubulin and βIII-tubulin were used as a loading control. X-Ray films were quantified by densitometry (OD). Newman-Keuls post-hoc test after ANOVA: * p<0.05, ** p<0.01, *** p<0.005 compare to control conditions. Plotted values are means +/- sem.

FIGS. 2. PAI-1 expression is increased by stress. Plasma Corticosterone levels (a), PAI-1, tPA, P-TrkB, P-Erk1/2^{MAPK}proteins measured by western blot (b, c) in the dorsal hippocampus of C57BL/6J mice in response to 30 min, 1 h (60 min) and 3 h (180 min) restraint stress. βIII-tubulin was used as a loading control. X-Ray films were quantified by densitometry (OD). Newman-Keuls post-hoc test after ANOVA: * p<0.05, *** p<0.005 compare to control conditions. Plotted values are means +/- sem.

FIGS. 3. The development of PTSD-like memory is associated with an increase in PAI-1 expression. Fear responses, expressed as % of time spent freezing, twenty-four hours after conditioning in C57BL/6J mice exposed in a safe environment to the tone not predicting the threat (non-predicting cue, a, b) or to the environment in which the conditioning was performed (predicting context, c, d). Expression of P-Erk1/2^{MAPK} (e, f) and PAI-1 (g, h) proteins in the dorsal hippocampus at different times after the conditioning sessions (e, g) and expressed as area under the curve encompassing the 24 h of analysis (f, h). Example of the western blot used for protein quantification by densitometry after normalization with the level of βIII-tubulin (i). Immediately after the conditioning session animals received an injection of either vehicle (Veh; NaCl 0.9% i.p., white symbol) or Cort (2 mg/kg i.p., black symbol). Grey symbol: control animals that were manipulated but not exposed to conditioning. Magnitude of tone conditioning represented by a normalized ratio: (tone - ((pre + post)/2)) / (tone + ((pre + post)/2)) (b). Student’s t-test and Fisher’s PLSD test after ANOVA: * p<0.05, ** p <0.01, *** p <0.005 vs Veh group. Plotted values are means +/- sem.

FIGS. 4. Effects of different doses of PAI-1 on PTSD-like memory. Fear responses, expressed as % of time spent freezing, twenty-four hours after conditioning in C57BL/6J mice exposed in a safe environment to the tone not predicting the threat (non-predicting cue, a, c) or to the environment in which the conditioning was performed (predicting context, b, d). Immediately after the conditioning session animals received one of the following treatments: an injection of vehicle (Veh; NaCl 0.9% i.p., white symbol); an injection of Cort (2 mg/kg i.p., black symbol); an intra-hippocampal infusion of PAI-1 (30, 90 or 240 ng/side, symbols with different shadows of gray/striped gray). Magnitude of tone conditioning represented by a normalized ratio: (tone - ((pre + post)/2)) / (tone + ((pre + post)/2)) (c). Post-hoc Newman-Keuls test after ANOVA: * p <0.05, *** p <0.005 vs Veh/Veh group. Plotted values are means +/- sem.

FIGS. 5. The increase in PAI-1 is a sufficient and necessary condition for the induction of PTSD-like memory. Fear responses, expressed as % of time spent freezing, twenty-four hours after conditioning in C57BL/6J mice exposed in a safe environment to the tone not predicting the threat (non-predicting cue, a, b, e, f) or to the environment in which the conditioning was performed (predicting context, c, d, g, h). Immediately after the conditioning session animals received one of the following treatments: an injection of (white symbol) vehicle (Veh; NaCl 0.9% i.p.) alone or in combination with the intra-hippocampal infusion of either (striped dark gray symbol) PAI-1 (240 ng/side) or (light gray symbol) PAI-1 (240 ng/side) + mature BDNF (100 ng/side); an injection of (black symbol) Cort alone (2 mg/kg i.p.) or in combination with the intra-hippocampal infusion of either the PAI-1 antagonist Tiplaxtinin (5 ng/side, dark gray symbol) or the vehicle of Tiplaxtinin (light gray symbol). Magnitude of tone conditioning represented by a normalized ratio: (tone - ((pre + post)/2)) / (tone + ((pre + post)/2)) (b and f). Post-hoc Newman-Keuls test after ANOVA: *** p<0.005 vs Veh/Veh group, ### p<0.005 vs Veh/PAI-1 group and * p <0.05, *** p <0.005 vs Veh group. Plotted values are means +/- sem.

FIGS. 6. PAI-1 expression is increased by stress. (a) Scheme of the experimental protocol. (b) PAI-1 level measured by ELISA in the plasma of C57BL/6J mice in response to 1 h (60 min, gray symbol) and 3 h (180 min, black symbol) restraint stress. * p<0.05, *** p<0.005 compare to control condition. Plotted values are means +/- sem

FIG. 7. PAI-1 blood level of French soldiers deployed in Afghanistan during a 6 month mission (n=14, control and n=11, PTSD+).

### EXAMPLE 1

### Material & Methods

Chemicals. In all the experiments we used a preformed water-soluble complex of corticosterone and 2-hydroxypropyl-β-cyclodextrin (#C174, Sigma, USA) (14-16). In mice, corticosterone (Cort; 2 mg/kg in a volume of 0.1 ml/10 g body weight) or vehicle (NaCl 0.9%) were administered i.p. immediately after the acquisition of fear conditioning in order to mimic the effect of intense trauma (5). Cort was used at 100 nM and 1000 nM on rat PC12 cells and at 10 nM and 1000 nM on rat hippocampal slices (14;16). Millipore (USA) provided the recombinant human BDNF (CAS Nb 218441-99-7, #GF029, 100 ng/side) and tPA inhibitor; stable recombinant mutant of human Type 1 Plasminogen Activator Inhibitor (PAI-1; CAS Nb: 140208-23-7, #528208, ranging from 30 to 240 ng/side) (16;18). The small-molecule inhibitor of PAI-1 activity Tiplaxtinin (PAI-039; CAS Nb: 393105-53-8, #1383, 5 ng/side) was provided by Axon MEDCHEM (The Netherlands).

Cell culture. PC12 cell line (ATCC CRL-1721) derived from a transplantable rat pheochromocytoma was used (14;16). PC12 cells were seeded on six well plates coated with poly-D-lysine at the appropriate concentration (10⁵ cells/well) in fresh, antibiotic-free medium (DMEM/F12 (#31330-038, Gibco, USA) + 10% Foetal Bovine Serum (FBS; #10270106, Fisher Scientific, USA)). Sixteen hours before Cort treatment, the medium was changed for a steroid-free culture medium (DMEM/F12 + 10% Charcoal/Dextran-treated FBS (#SH30068-03, Hyclone, Fisher Scientific, USA). PC12 cells (n=6/group) were treated with 100 nM and 1000 nM of corticosterone-HBC (Sigma, USA) then harvested after 3 h and the proteins and RNA extracted.

Hippocampal slice preparations and corticosterone treatment Hippocampal slice preparations have been described in detail previously (19). Briefly, adult male Sprague-Dawley rats (2-3 months old n=18, Charles River Laboratory, France) were used. Rats were then anesthetized with isoflurane and transcardially perfused with nearly frozen modified artificial cerebrospinal fluid (CSF) with 3 mM kynurenic acid. The modified CSF for perfusion contained: (in mM) 87 NaCl, 75 Sucrose, 25 Glucose, 5 KCl, 21 MgCl2, 0.5 CaCl₂ and 1.25 NaH₂PO₄. After perfusion, the brains were quickly removed and sliced (300 µm) in the coronal plane using a vibratome (Campden Instruments, UK). Immediately after cutting, slices were stored for 40 min at 32° C. in CSF ((in mM): 130 NaCl, 11 Glucose, 2.5 KCl, 2.4 MgCl₂, 1.2 CaCl₂, 23 NaHCO₃, 1.2 NaH₂PO₄), equilibrated with 95% O₂ / 5% CO₂ then stored at room temperature for the rest of the experiment. Each brain slice was then treated for 1 h (60 min) and 3 h (180 min) with 10 nM and 1000 nM of Cort. One slice served as a control reference and did not undergo any treatment. Dorsal hippocampi were isolated, and proteins were extracted as previously described (14;16).

Protein extraction from brain tissues and immunoblotting analysis. A detailed description of protein extraction and immunoblotting analysis has been reported previously (14-16;20;21). Briefly, protein sample extracts from PC12 cells and mouse and rat hippocampi were performed in RIPA buffer containing protease and phosphatase inhibitors (#P8340 and #P0044, Sigma, USA) before being subjected to immunoblotting experiments. SDS-PAGE-separated proteins were then revealed with relevant antibodies. Rabbit polyclonal anti-PAI-1 antibodies were from Lifespan Biosciences (LSBio#C81062, 1/1000, WA, USA) and Epitomics (#3917-1, 1/3000, CA, USA), anti-tPA (#T5600-05G; 1/5000) was from US Biologicals (MA, USA), anti-Erk1/2^{MAPK} (#06-182; 1/50000) was from Millipore (MA, USA), anti-Phospho-Erk1/2^{MAPK} (#9101S; 1/1000) was from CST (MA, USA). Rabbit monoclonal antibodies anti-Phospho-Erk1/2^{MAPK} (#4370; 1/5000) was from CST (MA, USA), anti-Phospho-TrkB (#2149-1; 1/5000) was from Epitomics (CA, USA), anti-TrkB (#610101; 1/2000) was from BD Biosciences (NJ, USA). Mouse monoclonal anti-Neuronal Class III β-Tubulin (TUJ1) (#MMS-435P; 1/20000) was from Eurogentec (Belgium), anti-α-tubulin (#N356, 1/50000) was from Amersham Life Sciences (Del, USA). CST provided secondary antibodies: anti-rabbit IgG, HRP-linked antibody (#7074, 1/5000) and anti-mouse IgG, HRP-linked antibody (#7076, 1/20000). In all experiments, βIII-tubulin or α-tubulin measures were used as a loading control. X-Ray films (Kodak, USA) were quantified by densitometry (optical density; OD) using a GS-800 scanner coupled with Quantity One software (Bio-Rad, CA, USA).

Quantitative PCR analysis. Samples of PC12 cells treated with Cort were homogenized in Tri-reagent (Euromedex, France) and RNA was isolated using a standard chloroform/isopropanol protocol (22). RNA was processed and analyzed using an adapted version of published methods (23). cDNA was synthesized from 2 µg of total RNA using RevertAid Premium Reverse Transcriptase (Fermentas, Thermo Fisher Scientific, USA) and primed with oligo-dT primers (Fermentas, Thermo Fisher Scientific, USA) and random primers (Fermentas, Thermo Fisher Scientific, USA). qPCR was perfomed using a LightCycler® 480 Real-Time PCR System (Roche, Meylan, France). qPCR reactions were done in duplicate for each sample, using transcript-specific primers, cDNA (4 ng) and LightCycler 480 SYBR Green I Master (Roche) in a final volume of 10 µl. The PCR data were exported and analyzed in a computer-based tool (Gene Expression Analysis Software Environment) developed at the Neurocentre Magendie (France). The Genorm method was used to determine the reference gene. Relative expression analysis was corrected for PCR efficiency and normalized against two reference genes. The ribosomal protein L13a (Rpl13a) and non-POU-domain-containing (Nono) genes were used as reference genes. The relative level of expression was calculated using the comparative (2^{-ΔΔCT}) method (24). qPCR amplification used specific primers to specifically amplify Serpine1 gene encoding PAI-1 protein and Nono and Rpl13a as reference genes.

PAI-1 forward primer sequence (5′-3′): GGCACAATCCAACAGAGACAATC and reverse primer sequence (5′-3′): AGGCTTCTCATCCCACTCTCAA. (SEQ ID N°3 and SEQ ID N°4) Restraint Stress. Male C57BL/6J mice aged 2-3 months old (n=30) were obtained from Charles River Laboratory, France. Mice were placed into 50 ml conical centrifuge tubes fitted with a central puncture so as to allow ventilation. The tubes were placed in horizontal holders with strong light exposure, and the animals were held in this way for a continuous period of restraint. After 30 min, 1 h (60 min) and 3 h (180 min) of restraint, the mice and those from an unstressed control group were sacrified by decapitation, then the hippocampi and blood were collected and assayed for protein extraction (16;21).

Blood collection for corticosterone assay. Blood was rapidly collected in heparine-EDTA-coated tubes (Sarstedt, France) and centrifuged at 2,000 rpm (4° C., 20 min). Supernatant containing the blood plasma was stored at -20° C., and then processed for corticosterone assay. Plasma corticosterone (Corticosterone EIA kit #KO14-H1, Arbor Assays, Michigan, USA) levels were quantified by ELISA following the manufacturer’s instructions (16;21).

### Behavioral Procedure.

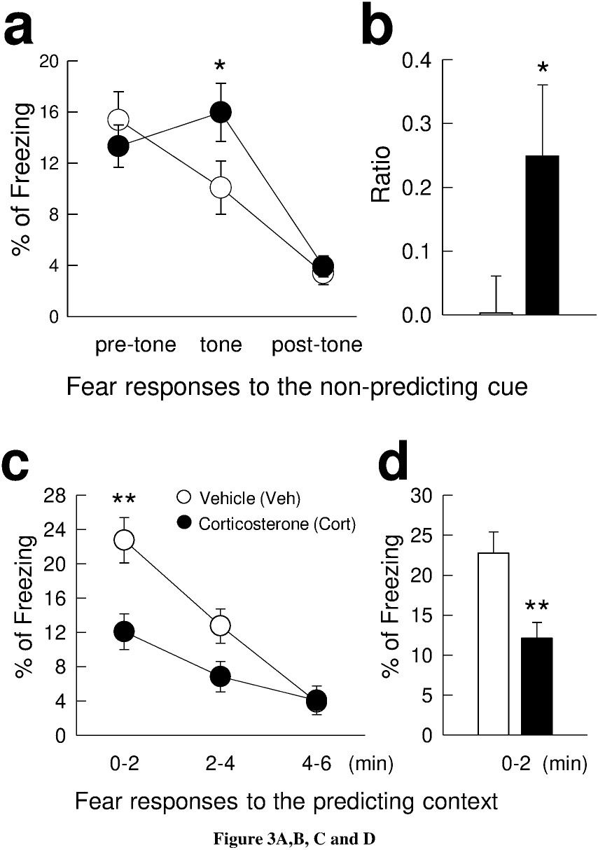

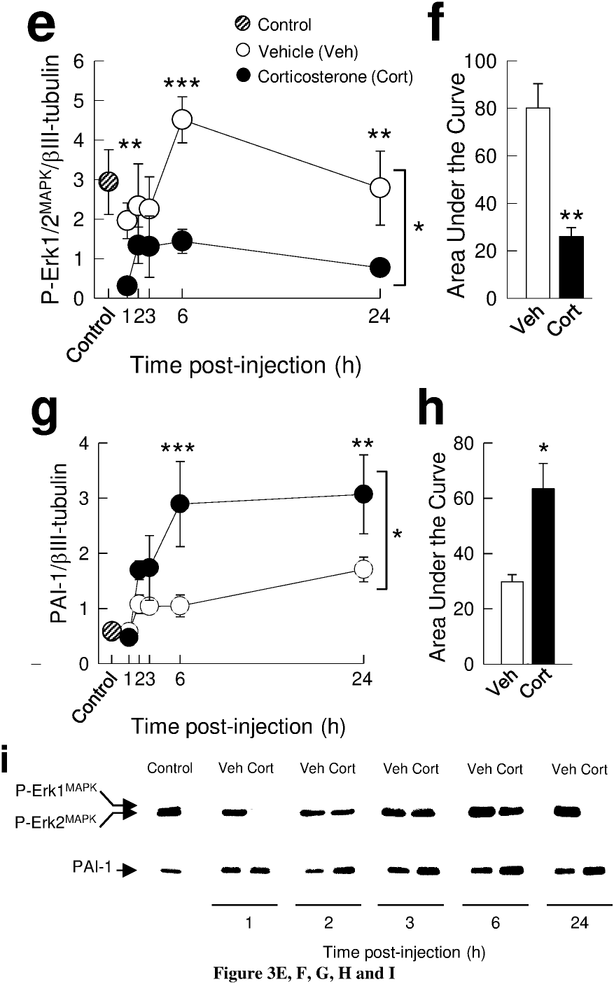

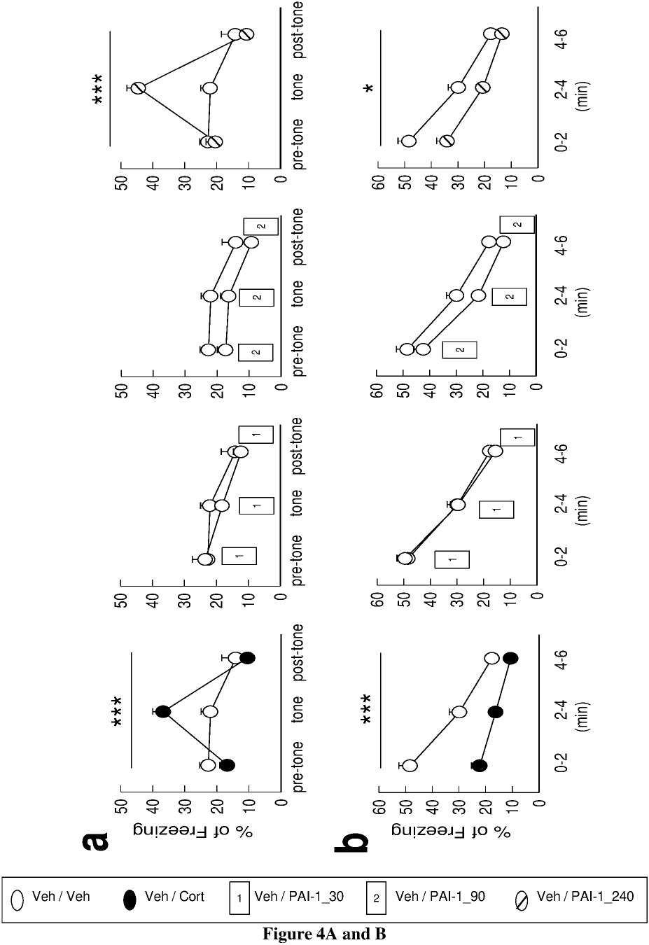

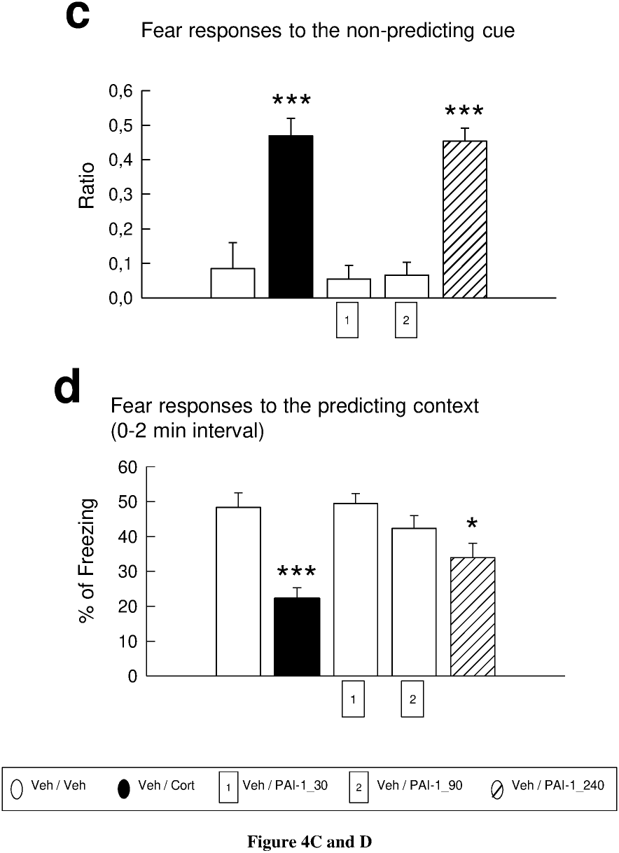

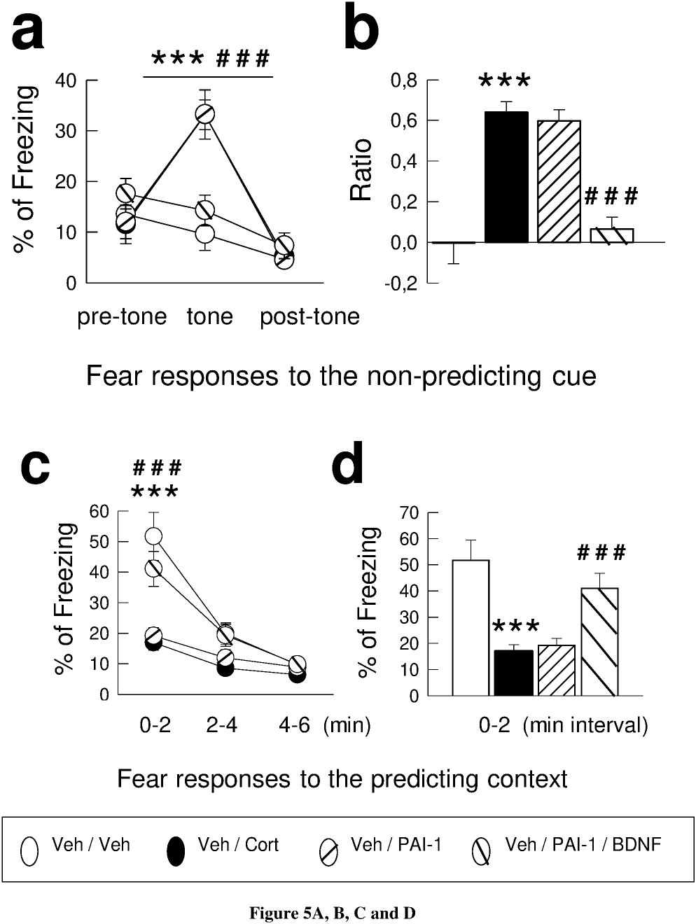

Surgical procedure. Male C57BL/6J mice {n=20 in Experiment 1 (FIGS. 3a-d), n=75 in Experiment 2 (FIG. 4), n=40 in Experiment 3 (FIGS. 5a-d) and n=48 in Experiment 4 (FIGS. 5e-h)} 3-4 months old (Charles River Laboratory, France) were used. Mice were surgically-implanted bilaterally 1 mm above the dorsal hippocampus (A/P, -2 mm; M/L, ±1.3 mm; D/V, 0.9 mm; relative to dura and bregma) following to Franklin and Paxinos’s mouse brain atlas (25) then allowed to recover for 8 days before the behavioral experiments.

Adaptive vs maladaptive (PTSD-like) fear memory. The behavioral model based on a general fear conditioning procedure has been fully described in a previous study (5).
Pre-exposure - The day before fear conditioning, each mouse was placed individually in an opaque PVC chamber (30 × 24 × 22 cm) with an opaque PVC floor, for 2 min, in a brightness of 10 lux. This pre-exposure allowed the mice to acclimate and become familiar with the chamber used for the cue alone test (“safe context”).
Induction of adaptive vs PTSD-like fear memory - Acquisition of fear conditioning was performed in a different context, consisting in a Plexiglas conditioning chamber (30 × 24 × 22 cm) with the floor connected to a shock generator, in a brightness of 110 lux, giving access to the different visual-spatial cues in the experimental room. Briefly, each animal placed in the conditioning chamber for 4 min received 2 footshocks (0.4 mA, 50 Hz, 1 s), which never co-occurred with 2 tone deliveries (70 dB, 1 kHz, 15 s). This tone-shock unpairing paradigm is known to make the contextual cues the primary stimuli that become associated with the footshock (5;26-28). Consequently, the phasic tone, although salient, is not predictive of the shock delivery, whereas the static contextual cues constitute the main predictor of the shock. Immediately after the acquisition of fear conditioning, mice received a systemic injection of either NaCl or Cort (see below for details). An adaptive fear memory will therefore be attested in control (NaCl-injected) mice by the expression of highly conditioned fear when re-exposed to the conditioning context and no conditioned fear when re-exposed to the irrelevant tone cue (in the safe context). In contrast, Cort-injected mice will display a maladaptive (PTSD-like) memory attested by an abnormally high fear response to the irrelevant tone (cue-based hypermnesia) together with a decreased conditioned fear to the conditioning context (contextual amnesia) (5).
Memory tests - After fear conditioning, each animal was returned to its home cage and 24 h later, all mice were submitted to two memory tests during which freezing behavior, defined as a lack of any movement except for respiratory-related movements (29), was measured and used as an index of conditioned fear. During these two memory tests, animals were continuously recorded for off-line second-by-second scoring of freezing by an observer blind to the experimental groups. Mice were first re-exposed to the tone within the “safe” context during which three successive recording sessions of the behavioral responses were performed: one before (first 2 min), one during (next 2 min), and one after (last 2 min) tone presentation. Conditioned response to the tone is expressed by the percentage of freezing during the tone presentation compared to the levels of freezing expressed before and after tone presentation (repeated measures on 3 blocks of freezing). The strength and specificity of this conditioned fear is attested by a ratio that represents the increase in the percentage of freezing with the tone with respect to a baseline freezing level {i.e., pre- and post-tone periods mean: (tone - ((pre + post)/2)) / (tone + ((pre + post)/2))}. Then two hours later, mice were re-exposed to the conditioning context alone for 6 min (without the tone cue). Freezing to the context was calculated as the percentage of the total time spent freezing during the successive three blocks of 2-min periods of the test. While the first block is the critical block attesting difference between animals that are conditioned to the conditioning context and those that are not or less, the following two blocks are presented in order to assess a gradual extinction of the fear responses in the absence of shock.
Molecular analysis - In the experiment (FIGS. 3e-i) measuring the modulation of GC-mediated PAI-1 expression and Erk1/2^{MAPK} signaling pathway after the acquisition of fear conditioning, separate groups (n=6-8 per group) of C57BL6J mice were sacrificed 1 h, 2 h, 3 h, 6 h and 24 h after the acquisition of fear conditioning and Cort or vehicle (NaCl 0.9%) injection. Naive C57/BL6J mice (n=15) are used to quantify basal protein expression levels. Dorsal hippocampi were then collected and assayed for immunoblotting analysis.

Drug injections. Immediately after the acquisition of fear conditioning, mice were randomly divided into groups firstly according to first their systemic injection of Cort and secondly their specific intra-hippocampal infusion. Cort (2 mg/kg in a volume of 0.1 ml/10 g body weight) or vehicle (NaCl 0.9%) was administered i.p. (5) while PAI-1, mature BDNF and Tiplaxtinin were intra-hippocampally infused.
Experiment 1 (FIGS. 3a-d): 1. Vehicle (NaCl 0.9%, n=8), 2. Cort (2 mg/kg, n=12).
Experiment 2 (FIGS. 4): 1. Vehicles (aCSF + NaCl 0.9%, n=15), 2. Cort (2 mg/kg + aCSF, n=15), PAI-1 (30 ng/side + NaCl 0.9%, n=15), PAI-1 (90 ng/side + NaCl 0.9%, n=15), PAI-1 (240 ng/side + NaCl 0.9%, n=15).
Experiment 3 (FIGS. 5a-d): 1. Vehicles (aCSF + NaCl 0.9%, n=8), 2. Cort (2 mg/kg + aCSF, n=8), PAI-1 (240 ng/side + NaCl 0.9%, n=12), PAI-1 + mature BDNF (240 ng/side + 100 ng/side, respectively + NaCl 0.9%, n=12).
Experiment 4 (FIGS. 5e-h): 1. Vehicles (aCSF + DMSO, n=12), 2. Cort (2 mg/kg + DMSO, n=12), Tiplaxtinin (5 ng/side + NaCl 0.9%, n=12), Tiplaxtinin + Cort (5 ng/side + 2 mg/kg, respectively, n=12).

PAI-1 and BDNF were diluted in artificial CSF (aCSF) and Tiplaxtinin in 1.6 % dimethyl sulfoxide (DMSO) and then diluted in aCSF. Bilateral infusions of 0.3 µl/side were administered into the dorsal hippocampus immediately after acquisition of fear conditioning at a constant rate (0.1 µl/min).

Histology. A detailed description of the histological protocol was reported previously (14;15;30). Briefly, after completion of the behavioral study, animals were sacrificed in order to evaluate the cannulae placements.

Data analysis. All experiments involving mice and rats were performed according to the protocols approved by the Aquitaine-Poitou Charentes local ethical committee (authorization number APAF1S#7397-20161 02814453778 v2) in strict compliance with the French Ministry of Agriculture and Fisheries (authorization number D33-063-096) and European Communities Council Directive (2010/63/EU). All efforts were made to minimize animal suffering and to reduce the number of rodents used, while maintaining reliable statistics. All experiments were conducted with experimenters blind to drug treatment conditions; no randomization method for the constitution of the experimental groups was applied. The sample size was chosen to ensure adequate statistical power for all experiments. Statistical analyses were performed using analysis of variance (ANOVA) followed by either Newman Keuls or Fisher’s PLSD post-hoc test for pairwise comparisons. Student’s t-test was used for pairwise comparisons. A significance level of p<0.05 was used for all statistical analyses. Statistical significance was expressed as * = P<0.05; ** = P<0.01; *** or ### = P<0.005. All values were expressed as mean ± s.e.m.

### Results

### Corticosterone and Stress Stimulated the Expression of PAI-1 Protein

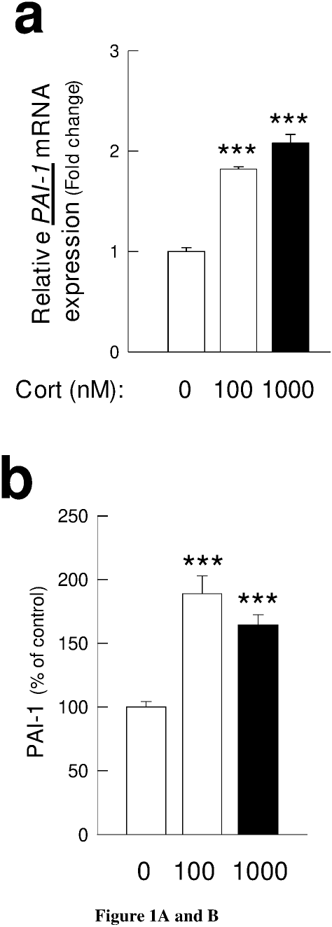

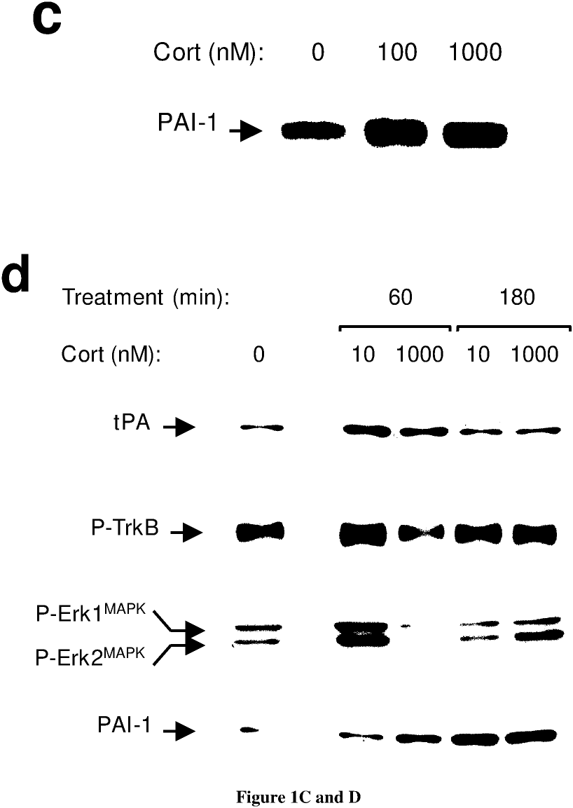

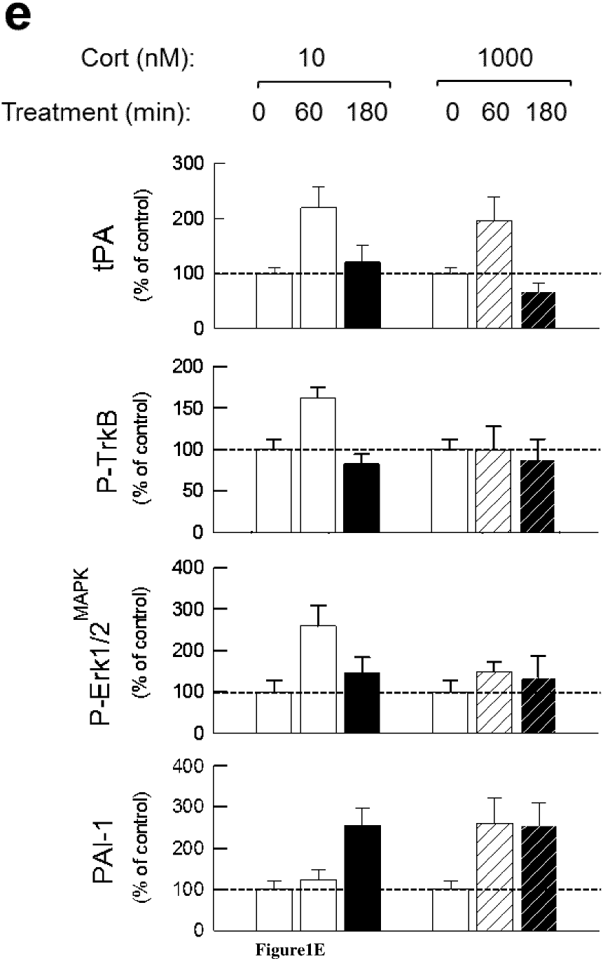

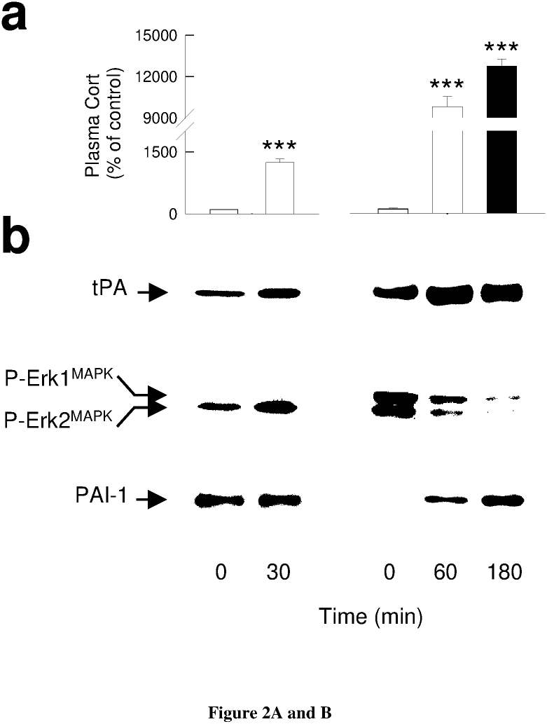

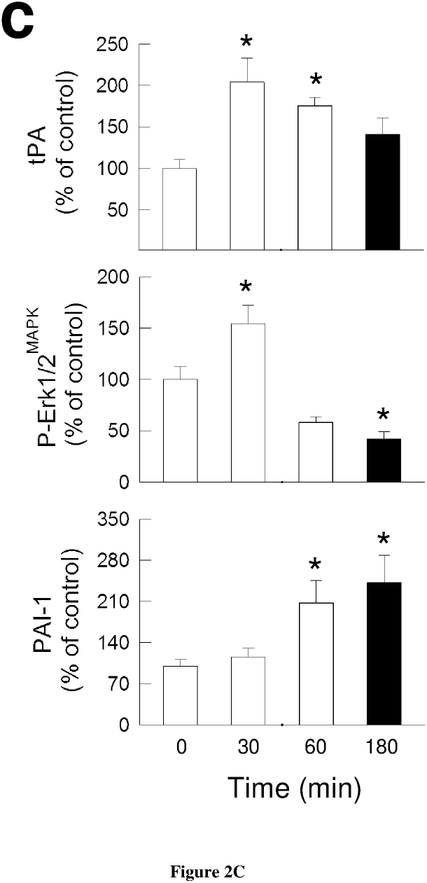

Our previous reports have revealed that the tPA/plasmin system induced by GC-activated GR is a core effector in the regulation of the pro-BDNF/BDNF balance allowing, through the activation of the TrkB/Erk1/2^{MAPK} signaling cascade, the formation of normal fear memory (14-16). Interestingly, the major potential physiological inhibitor of this pro-memory cascade, the tPA inhibitor PAI-1 (Type 1 Plasminogen Activator Inhibitor), also displays Glucocorticoid Responsive Elements (GRE) in the regulatory sequences of its gene (31). Since the effects of GC and stress on PAI-1 are unknown, in a first experiment we treated PC12 cells, which expresses both endogenous GR and PAI-1 (14;32), with corticosterone (Cort) the major GC in rodents. After 3 h of treatment, Cort (100 and 1000 nM) strongly increased the expression of PAI-1 mRNA (FIG. 1a) and protein (FIGS. 1b, c). In a second experiment we then assessed the expression of PAI-1 in the hippocampus, the major target of the GC effect on memory (FIGS. 1d, e). In hippocampal slices, concentrations of Cort (10 nM), mimicking moderate stress conditions, induced first an increase in tPA, P-TrkB and P-Erk1/2^{MAPK} (1 h after treatment) followed (3 h after treatment) by an increase in PAI-1. The increase in PAI-1 at 3 h was associated with the return of tPA, P-TrkB and P-Erk1/2^{MAPK} at basal levels (FIGS. 1d, e). In contrast, high concentrations of Cort (1000 nM) induced an early increase in PAI-1 (1 h after treatment) which was also accompanied by the suppression of the increase in memory-promoting proteins tPA, P-TrkB and P-Erk1/2^{MAPK} after 3 h treatment (FIGS. 1d, e). Combining in vitro and ex vivo approaches, GR-expressing cell lines and hippocampal slices respectively, we identified PAI-1 as a plausible upstream molecular effector activated by increasing amounts of GC. Since the secretion of Cort increases systemically in response to stress (20), in a third experiment we studied the effects of different stress intensities on the expression of tPA and PAI-1 in the hippocampus of C57BL/6J mice. We compared 30 min, 1 h and 3 h of restraint stress which induces progressively higher plasma levels of corticosterone (FIG. 2a). Strikingly after 30 min of moderate stress only the memory-promoting proteins tPA and P-Erk1/2^{MAPK} were increased (FIGS. 2b, c). In contrast, in more intense stress conditions (1 h and 3 h) there was a strong increase in PAI-1 associated with the inhibition of P-Erk1/2^{MAPK} which progressively went below basal levels (FIGS. 2b, c). The results of this first series of experiments suggest that a moderate increase in GC concentrations during stress first triggers the activation of the memory-promoting tPA/TrkB/Erk1/2^{MAPK} molecular cascade and later on of its inhibitor PAI-1. However, during intense stress a high level of GC induces early activation of PAI-1 which inhibits the memory-promoting tPA/TrkB/Erk1/2^{MAPK} molecular cascade.

### PAI-1 Protein is a Sufficient Condition to Induce PTSD-Like Memories

In a second series of experiments, we investigated whether changes in the expression of PAI-1 could determine the appearance of PTSD-like memories, which were evaluated using a previously described mouse model (5). Mice were submitted to a threatening situation - the delivery of an electric foot shock - when exposed to a specific context (conditioning cage). A discrete cue (a tone) was also repeatedly presented during conditioning but was never paired with shock delivery. In these conditions the context is the correct predictor of the threat (predicting context), whilst the cue although present with the threat does not predict it (non-predicting cue). Twenty-four hours after this conditioning procedure, animals were re-exposed first to the cue alone in a familiar and safe environment and then to the conditioning context without the cue (5). In control conditions Veh-injected mice showed a fear response (freezing) when exposed to the correct predictor of the threat, the predicting context, but not when exposed to the non-predicting cue (FIGS. 3a-d). However, if mice were injected with Cort (2 mg/kg) immediately after conditioning, as previously described (5), PTSD-like memory impairments appeared. In this case, mice did not show fear in response to the correct predictor of the threat, the predicting context, but in response to the non-predicting tone (FIGS. 3a-d). Like PTSD patients, mice injected with Cort lost the ability to restrict fear to the right situation or cue (9). Using this model, we first compared the expression of P-Erk1/2^{MAPK} and PAI-1 in the dorsal hippocampus (FIGS. 3e-i. In control mice, showing normal fear memory, the concentrations of the memory-promoting proteins P-Erk1/2^{MAPK} progressively increased after the conditioning session, whilst it decreased below basal levels in animals that developed PTSD-like memories (FIGS. 3e, f, i). The opposite pattern was observed for PAI-1 which reached much higher concentrations in animals showing PTSD-like memories than in control mice (FIGS. 3g, h, i). In a second experiment, we assessed whether this increase in PAI-1 was a sufficient condition for inducing PTSD-like memories. For this purpose, after conditioning, we injected different concentrations of PAI-1 into the dorsal hippocampus (FIG. 4). Similarly, to what was observed after Cort, PAI-1 (240 ng/side) induced PTSD-like memory with animals showing fear in response to the non-predicting cue but not to the predicting-context. The results of this second series of experiments suggest that the increase in PAI-1 triggered by GC is a sufficient condition to induce PTSD-like memories.

### PAI-1 Protein is a Necessary Condition to Induce PTSD-Like Memories

In the third series of experiments, we wanted to establish whether it was possible to block the development of PTSD-like memory. To address this issue, we first assessed the hypothesis whether PTSD-like memory induced by injection of PAI-1 (FIG. 4) is rescued by infusion of mature BDNF in the dorsal hippocampus. Mature BDNF should be sufficient to bypass the PAI-1 inhibitory effect on the tPA/plasmin system to allow normal fear memory. Indeed the effect of PAI-1 was completely reversed by the concomitant injection of mature BDNF (FIGS. 5a-d), which indicates that PAI-1 likely induces PTSD-like memory by blocking the tPA-mediated proteolytic processing of pro-BDNF to mature BDNF (33). These evidences suggest that inhibiting hippocampal PAI-1 could be a valuable therapeutic strategy for the treatment of PTSD-like memory. Among the several PAI-1 inhibitors, Tiplaxtinin (PAI-039) has been well characterized in several animal models, demonstrating promise as a PAI-1 antagonist (34-36). In this experiment, we assessed whether an increase in PAI-1 was a necessary condition for the appearance of PTSD-like memories. We demonstrated that intra-hippocampal inhibition of PAI-1 by the injection of its antagonist Tiplaxtinin (PAI-039) immediately after the conditioning session prevented the appearance of PTSD-like memory in Cort-treated animals (FIGS. 5e-h).

Taken together the findings of these experiments indicated that an increase in PAI-1 levels triggered by high levels of GC is a sufficient and necessary condition to induce PTSD-like memories.

### Discussion

Uncovering the molecular mechanisms of the shift from beneficial to harmful effects of stress and GC is a key question in understanding the pathophysiological mechanism through which life events can induce psychiatric disorders. Our results are of major importance because they provide the first molecular signaling model in which the beneficial and harmful effects of stress and GC on a cognitive process as memory can be dissociated. We previously showed that in moderate stress conditions, GC hormones induce the expression of the tPA protein which by increasing the production of mature BDNF triggers the activation of the TrkB/Erk1/2^{MAPK} cascade which strengthens the memory trace of the stress-related event (14-16). Here we showed that the activity of the tPA/BDNF/TrkB/Erk1/2^{MAPK} cascade is then inhibited by the delayed production of the tPA inhibitor PAI-1. However, in the case of particularly stressful conditions and very high levels of GC, the production of PAI-1 is triggered early on. PAI-1 then blocks the activity of tPA and inhibits the pro-mnesic BDNF/TrkB/Erk1/2^{MAPK} signaling cascade, inducing PTSD-like memory. By lowering hippocampal PAI-1 activity, Tiplaxtinin (34-36) restored the formation of a hippocampal-dependent adaptive (“contextualized”) fear memory and thus normalizes traumatic memory.

Several lines of evidence support the involvement of impairment of BDNF processing mediated by PAI-1 in the pathophysiology of stress-related diseases. Firstly, impaired BDNF function has been associated with PTSD both in rodents and human (37). Secondly, PTSD patients have a higher risk of cardiovascular pathophysiologies and notably atherothrombosis (38), for which high levels of PAI-1 is a known risk factor (39). Thirdly, elevated PAI-1 levels and polymorphisms of the SERPINE1 gene encoding the PAI-1 protein have also been related to depression, another stress-induced condition (40;41).

An increase in PAI-1 could mediate the pathological effects of stress not only by decreasing the production of mature BDNF but also by promoting the accumulation of pro-BDNF that is no longer cleaved into mature BDNF by the tPA-activated plasmin. Indeed, although long considered to be inactive, pro-BDNF forms are able to form a ternary complex with the p75^{NTR} and sortilin receptors, to induce neuronal cell death by apoptosis (42). In addition, pro-BDNF/p75^{NTR} signaling has been shown to have the opposite effect on synaptic plasticity, inducing LTD whilst BDNF/TrkB signaling induces LTP (43). These results are consistent with brain imaging findings showing hippocampal atrophy reported in PTSD subjects (10).

Although the biphasic effects of activated GR have been described before in other contexts (44;45), the exact mechanism through which the dose-dependent effects of GC regulate the transcription of the PAI-1 encoding gene deserves further discussion. The PAI-1 promoter is known to have, in addition to GRE, response elements for the AP-1 transcription factor complex (Fos:Jun) (46). A plausible explanation of the observed dose-dependent effects is that when moderate levels of activated GR are produced, GR/AP1 heterodimers, which are known to promote reciprocal transcriptional interference, are mostly formed and prevent PAI-1 transcription through a protein-protein interaction mediated-sequestration process (47;48). In contrast, when high levels of activated GR are produced, for example after intense stress, GR/GR homodimers are now formed which are able to activate the transcription of the PAI-1 encoding gene.

In conclusion, our data show that the transition from adaptive to maladaptive stress-related memories is mediated by a shift in balance between tPA and PAI-1 proteins, with an adaptive increase in memory appearing when the ratio is in favor of tPA (16) and PTSD-like memory when it is in favor of PAI-1. As a consequence, PAI-1 levels after a traumatic event could be a predictive biomarker of the appearance of PTSD and pharmacological inhibition of PAI-1 activity a new therapeutic approach of this debilitating condition.

### EXAMPLE 2 PAI BLOOD LEVEL IN RESPONSE TO RESTRAIN STRESS

### Materials and Methods

Blood collection for PAI-1 assay. Blood was rapidly collected in heparine-EDTA-coated tubes (Sarstedt, France) and centrifuged at 2,000 rpm (4° C., 20 min). Supernatant containing the blood plasma was stored at -20° C., and then processed for PAI-1 assay. PAI-1 (Murine PAI-1 total antigen assay #MPAIKT-TOT, Molecular Innovation, Michigan, USA) expression levels were quantified by ELISA following the manufacturer’s instructions

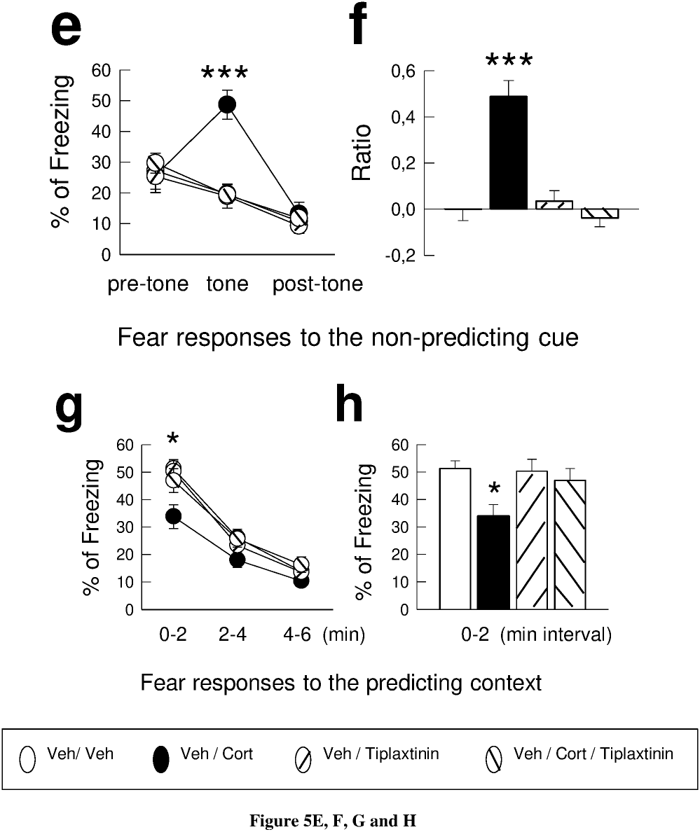

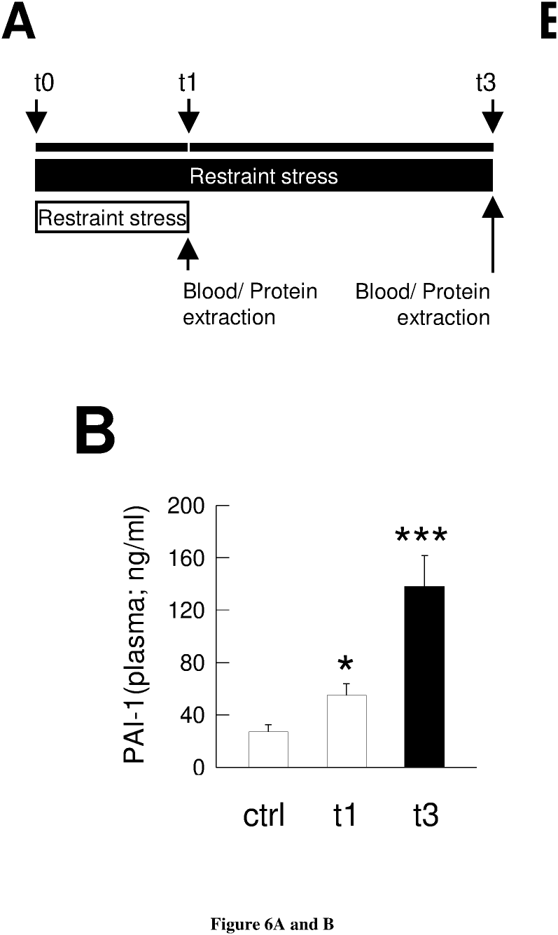

Since PAI-1 circulates in the blood and was shown to be upregulated upon stress (FIGS. 2), it represents an interesting candidate as biomarker of stress susceptibility, notably to assess its potential as a biomarker of PTSD. To test this hypothesis, we compared 1 h and 3 h of restraint stress in mice which induces progressively higher plasma levels of corticosterone (FIG. 2a). Indeed after 1 h and 3 h of intense stress conditions we showed a progressive and strong increase in PAI-1 blood level (FIGS. 6).

### Table Section

TABLE 1

Useful nucleotide and amino acid sequences for practicing the invention

| | |
|---|---|
| SEQ ID NO | Nucleotide and amino acid sequences |
| 1 (Human PAI-1 AA sequence) | MQMSPALTCLVLGLALVFGEGSAVHHPPSYVAHLASDFGVR VFQQVAQASKDRNVVFSPYGVASVLAMLQLTTGGETQQQIQ AAMGFKIDDKGMAPALRHLYKELMGPWNKDEISTTDAIFVQ RDLKLVQGFMPHFFRLFRSTVKQVDFSEVERARFIINDWVKTH TKGMISNLLGKGAVDQLTRLVLVNALYFNGQWKTPFPDSSTH RRLFHKSDGSTVSVPMMAQTNKFNYTEFTTPDGHYYDILELP YHGDTLSMFIAAPYEKEVPLSALTNILSAQLISHWKGNMTRLP RLLVLPKFSLETEVDLRKPLENLGMTDMFRQFQADFTSLSDQE PLHVAQALQKVKIEVNESGTVASSSTAVIVSARMAPEEIIMDR PFLFVVRHNPTGTVLFMGQVMEP |
| 2 (Human PAI-1 nucleic acid sequence) | acagctgtgt ttggctgcag ggccaagagc gctgtcaaga agacccacac gcccccctcc agcagctgaa ttcctgcagc tcagcagccg ccgccagagc aggacgaacc gccaatcgca aggcacctct gagaacttca ggatg cagat gtctccagcc ctcacctgcc tagtcctggg cctggccctt gtctttggtg aagggtctgc tgtgcaccat cccccatcct acgtggccca cctggcctca gacttcgggg tgagggtgtt tcagcaggtg gcgcaggcct ccaaggaccg caacgtggtt ttctcaccct atggggtggc ctcggtgttg gccatgctcc agctgacaac aggaggagaa acccagcagc agattcaagc agctatggga ttcaagattg atgacaaggg catggccccc gccctccggc atctgtacaa ggagctcatg gggccatgga acaaggatga gatcagcacc acagacgcga tcttcgtcca gcgggatctg aagctggtcc agggcttcat gccccacttc ttcaggctgt tccggagcac ggtcaagcaa gtggactttt cagaggtgga gagagccaga ttcatcatca atgactgggt gaagacacac acaaaaggta tgatcagcaa cttgcttggg aaaggagccg tggaccagct gacacggctg gtgctggtga atgccctcta cttcaacggc cagtggaaga ctcccttccc cgactccagc acccaccgcc gcctcttcca caaatcagac ggcagcactg tctctgtgcc catgatggct cagaccaaca agttcaacta tactgagttc accacgcccg atggccatta ctacgacatc ctggaactgc cctaccacgg ggacaccctc agcatgttca ttgctgcccc ttatgaaaaa gaggtgcctc tctctgccct caccaacatt ctgagtgccc agctcatcag ccactggaaa ggcaacatga ccaggctgcc ccgcctcctg gttctgccca agttctccct ggagactgaa gtcgacctca ggaagcccct agagaacctg ggaatgaccg acatgttcag acagtttcag gctgacttca cgagtctttc agaccaagag cctctccacg tcgcgcaggc gctgcagaaa gtgaagatcg aggtgaacga gagtggcacg gtggcctcct catccacagc tgtcatagtc tcagcccgca tggcccccga ggagatcatc atggacagac ccttcctctt tgtggtccgg cacaacccca caggaacagt |
| | ccttttcatg ggccaagtga tggaaccctg accctgggga aagacgcctt catctgggac aaaactggag atgcatcggg aaagaagaaa ctccgaagaa aagaatttta gtgttaatga ctctttctga aggaagagaa gacatttgcc ttttgttaaa agatggtaaa ccagatctgt ctccaagacc ttggcctctc cttggaggac ctttaggtca aactccctag tctccacctg agaccctggg agagaagttt gaagcacaac tcccttaagg tctccaaacc agacggtgac gcctgcggga ccatctgggg cacctgcttc cacccgtctc tctgcccact cgggtctgca gacctggttc ccactgaggc cctttgcagg atggaactac ggggcttaca ggagcttttg tgtgcctggt agaaactatt tctgttccag tcacattgcc atcactcttg tactgcctgc caccgcggag gaggctggtg acaggccaaa ggccagtgga agaaacaccc tttcatctca gagtccactg tggcactggc cacccctccc cagtacaggg gtgctgcagg tggcagagtg aatgtccccc atcatgtggc ccaactctcc tggcctggcc atctccctcc ccagaaacag tgtgcatggg ttattttgga gtgtaggtga cttgtttact cattgaagca gatttctgct tccttttatt tttataggaa tagaggaaga aatgtcagat gcgtgcccag ctcttcaccc cccaatctct tggtggggag gggtgtacct aaatatttat catatccttg cccttgagtg cttgttagag agaaagagaa ctactaagga aaataatatt atttaaactc gctcctagtg tttctttgtg gtctgtgtca ccgtatctca ggaagtccag ccacttgact ggcacacacc cctccggaca tccagcgtga cggagcccac actgccacct tgtggccgcc tgagaccctc gcgccccccg cgcccctctt tttccccttg atggaaattg accatacaat ttcatcctcc ttcaggggat caaaaggacg gagtgggggg acagagactc agatgaggac agagtggttt ccaatgtgtt caatagattt aggagcagaa atgcaagggg ctgcatgacc taccaggaca gaactttccc caattacagg gtgactcaca gccgcattgg tgactcactt caatgtgtca tttccggctg ctgtgtgtga gcagtggaca cgtgaggggg gggtgggtga gagagacagg cagctcggat tcaactacct tagataatat ttctgaaaac ctaccagcca gagggtaggg cacaaagatg gatgtaatgc actttgggag gccaaggcgg gaggattgct tgagcccagg agttcaagac cagcctgggc aacataccaa gacccccgtc tctttaaaaa tatatatatt ttaaatatac ttaaatatat atttctaata tctttaaata tatatatata ttttaaagac caatttatgg gagaattgca cacagatgtg aaatgaatgt aatctaatag aagcctaatc agcccaccat gttctccact gaaaaatcct ctttctttgg ggtttttctt tctttctttt ttgattttgc actggacggt gacgtcagcc atgtacagga tccacagggg tggtgtcaaa tgctattgaa attgtgttga attgtatgct ttttcacttt tgataaataa acatgtaaaa atgtttcaaa aaaataataa aataaataaa |
| 3) mouse PAI-1 forward primer sequence | ggcacaatccaacagagacaatc |
| 4) mouse PAI-1 reverse primer sequence | aggcttctcatcccactctcaa |

### EXAMPLE 3: EVALUATING PAI-1 BLOOD EXPRESSION LEVEL IN HUMAN WITH PTSD

We recently showed that under high stress conditions the downregulation of the GMES signaling cascade, through Glucocorticoid-mediated PAI-1 increase in the hippocampus, underlies the transition from a normal to PTSD-like fear memory impairment as the one observed in PTSD patients (49).

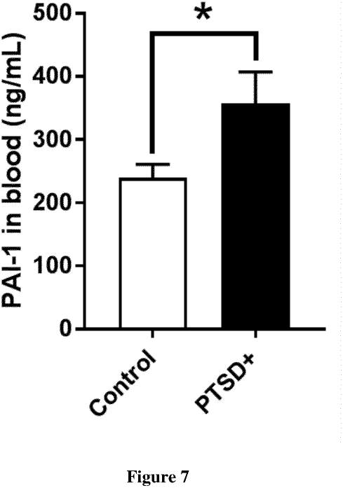

Therefore our current hypothesis suggests that PAI-1 could be a promising target for the diagnosis and therapy of PTSD. To assess this hypothesis, we conducted a promising pilot study in collaboration with Dr. M. Trousselard (Chief Medical Officer of the NSCo Department, and coordinator of the IRBA) who have collected blood sample from French soldiers after a six month mission in Afghanistan. Some soldiers developed PTSD (i.e. PTSD+) afterwards while others who experienced the same violence during the fights did not develop PTSD (i.e. Control). We used Enzyme-Linked ImmunoSorbent Assay method (i.e. ELISA) to quantify PAI-1 blood level. Our study revealed significant higher levels of PAI-1 in the blood of soldiers with PTSD, compared to non PTSD soldiers (t = 2.333 df = 23,p < 0.0287), collected after their deployment to the war zone (FIG. 7).

### REFERENCES

Throughout this application, various references describe the state of the art to which this invention pertains. The disclosures of these references are hereby incorporated by reference into the present disclosure.
(1) McEwen BS. The neurobiology of stress: from serendipity to clinical relevance. Brain Res 2000 Dec 15;886(1-2):172-89.
(2) De Kloet ER, Joels M, Holsboer F. Stress and the brain: from adaptation to disease. Nat Rev Neurosci 2005 Jun;6(6):463-75.
(3) Finsterwald C, Alberini CM. Stress and glucocorticoid receptor-dependent mechanisms in long-term memory: from adaptive responses to psychopathologies. Neurobiol Learn Mem 2014 Jul;112:17-29.
(4) Piazza PV, Le Moal M. The role of stress in drug self-administration. Trends Pharmacol Sci 1998 Feb;19(2):67-74.
(5) Kaouane N, Porte Y, Vallee M, Brayda-Bruno L, Mons N, Calandreau L, et al. Glucocorticoids Can Induce PTSD-Like Memory Impairments in Mice. Science 2012 Feb 23;335(6075):1510-3.
(6) Deppermann S, Storchak H, Fallgatter AJ, Ehlis AC. Stress-induced neuroplasticity: (mal)adaptation to adverse life events in patients with PTSD--a critical overview. Neuroscience 2014 Dec 26;283:166-77.
(7) American Psychiatric Association. Diagnostic and Statistical Manual of Mental Disorders (DSM-5). American PsychiatricPress, Washington D.C. ed. 2013.
(8) Kessler RC, Sonnega A, Bromet E, Hughes M, Nelson CB. Posttraumatic stress disorder in the National Comorbidity Survey. Arch Gen Psychiatry 1995 Dec;52(12): 1048-60.
(9) Desmedt A, Marighetto A, Piazza PV. Abnormal Fear Memory as a Model for Posttraumatic Stress Disorder. Biol Psychiatry 2015 Sep 1;78(5):290-7.
10) Sala M, Perez J, Soloff P, Ucelli di NS, Caverzasi E, Soares JC, et al. Stress and hippocampal abnormalities in psychiatric disorders. Eur Neuropsychopharmacol 2004 Oct;14(5):393-405.
(11) Rauch SL, Shin LM, Phelps EA. Neurocircuitry models of posttraumatic stress disorder and extinction: human neuroimaging research--past, present, and future. Biol Psychiatry 2006 Aug 15;60(4):376-82.
(12) Salehi B, Cordero MI, Sandi C. Learning under stress: the inverted-U-shape function revisited. Learn Mem 2010 Oct;17(10):522-30.
(13) Yerkes RM, Dodson JD. The relation of strength of stimulus to rapidity of habit formation. Journal of Comparative and Neurological Psychology 1908 Jan 1;18:459-82.
(14) Revest JM, Kaouane N, Mondin M, Le RA, Rouge-Pont F, Vallee M, et al. The enhancement of stress-related memory by glucocorticoids depends on synapsin-Ia/Ib. Mol Psychiatry 2010 Dec;15(12):1125, 1140-25, 1151.
(15) Revest JM, Di Blasi F, Kitchener P, Rouge-Pont F, Desmedt A, Turiault M, et al. The MAPK pathway and Egr-1 mediate stress-related behavioral effects of glucocorticoids. Nat Neurosci 2005 May;8(5):664-72.
(16) Revest JM, Le Roux A, Roullot-Lacarriere V, Kaouane N, Vallee M, Kasanetz F, et al. BDNF-TrkB signaling through Erk1/2 MAPK phosphorylation mediates the enhancement of fear memory induced by glucocorticoids. Mol Psychiatry 2014 Sep;19(9):1001-9.
(17) Weikum ER, Knuesel MT, Ortlund EA, Yamamoto KR. Glucocorticoid receptor control of transcription: precision and plasticity via allostery. Nat Rev Mol Cell Biol 2017 Mar;18(3):159-74.
(18) Nagai T, Kamei H, Ito M, Hashimoto K, Takuma K, Nabeshima T, et al. Modification by the tissue plasminogen activator-plasmin system of morphine-induced dopamine release and hyperlocomotion, but not anti-nociceptive effect in mice. J Neurochem 2005 Jun;93(5):1272-9.
(19) Kasanetz F, Lafourcade M, Deroche-Gamonet V, Revest JM, Berson N, Balado E, et al. Prefrontal synaptic markers of cocaine addiction-like behavior in rats. Mol Psychiatry 2013 Jun;18(6):729-37.
(20) Kitchener P, Di Blasi F, Borrelli E, Piazza PV. Differences between brain structures in nuclear translocation and DNA binding of the glucocorticoid receptor during stress and the circadian cycle. Eur J Neurosci 2004 Apr;19(7): 1837-46.
(21) Sarrazin N, Di Blasi F, Roullot-Lacarriere V, Rouge-Pont F, Le Roux A, Costet P, et al. Transcriptional effects of glucocorticoid receptors in the dentate gyrus increase anxiety-related behaviors. PLoS ONE 2009 Nov 2;4(11):e7704.
(22) Chomczynski P, Sacchi N. Single-step method of RNA isolation by acid guanidinium thiocyanate-phenol-chloroform extraction. Anal Biochem 1987 Apr;162(1):156-9.
(23) Bustin SA, Benes V, Garson JA, Hellemans J, Huggett J, Kubista M, et al. The MIQE guidelines: minimum information for publication of quantitative real-time PCR experiments. Clin Chem 2009 Apr;55(4):611-22.
(24) Livak KJ, Schmittgen TD. Analysis of relative gene expression data using real-time quantitative PCR and the 2(-Delta Delta C(T)) Method. Methods 2001 Dec;25(4):402-8.
(25) Paxinos G, Franklin KBJ. The mouse brain in stereotaxic coordinates, second edition. 2001.
(26) Calandreau L, Desmedt A, Decorte L, Jaffard R. A different recruitment of the lateral and basolateral amygdala promotes contextual or elemental conditioned association in Pavlovian fear conditioning. Learn Mem 2005 Jul;12(4):383-8.
(27) Calandreau L, Trifilieff P, Mons N, Costes L, Marien M, Marighetto A, et al. Extracellular hippocampal acetylcholine level controls amygdala function and promotes adaptive conditioned emotional response. J Neurosci 2006 Dec 27;26(52):13556-66.
(28) Calandreau L, Desgranges B, Jaffard R, Desmedt A. Switching from contextual to tone fear conditioning and vice versa: the key role of the glutamatergic hippocampal-lateral septal neurotransmission. Learn Mem 2010 Sep;17(9):440-3.
(29) Blanchard RJ, Blanchard DC. Crouching as an index of fear. J Comp Physiol Psychol 1969 Mar;67(3):370-5.
(30) Desmedt A, Garcia R, Jaffard R. Differential modulation of changes in hippocampal-septal synaptic excitability by the amygdala as a function of either elemental or contextual fear conditioning in mice. J Neurosci 1998 Jan 1;18(1):480-7.
(31) Bruzdzinski CJ, Johnson MR, Goble CA, Winograd SS, Gelehrter TD. Mechanism of glucocorticoid induction of the rat plasminogen activator inhibitor-1 gene in HTC rat hepatoma cells: identification of cis-acting regulatory elements. Mol Endocrinol 1993 Sep;7(9):1169-77.
(32) Vician L, Basconcillo R, Herschman HR. Identification of genes preferentially induced by nerve growth factor versus epidermal growth factor in PC12 pheochromocytoma cells by means of representational difference analysis. J Neurosci Res 1997 Oct 1;50(1):32-43.
(33) Pang PT, Teng HK, Zaitsev E, Woo NT, Sakata K, Zhen S, et al. Cleavage of proBDNF by tPA/plasmin is essential for long-term hippocampal plasticity. Science 2004 Oct 15;306(5695):487-91.
(34) Elokdah H, Abou-Gharbia M, Hennan JK, McFarlane G, Mugford CP, Krishnamurthy G, et al. Tiplaxtinin, a novel, orally efficacious inhibitor of plasminogen activator inhibitor-1: design, synthesis, and preclinical characterization. J Med Chem 2004 Jul 1;47(14):3491-4.
(35) Smith LH, Dixon JD, Stringham JR, Eren M, Elokdah H, Crandall DL, et al. Pivotal role of PAI-1 in a murine model of hepatic vein thrombosis. Blood 2006 Jan 1;107(1):132-4.
(36) Gerenu G, Martisova E, Ferrero H, Carracedo M, Rantamaki T, Ramirez MJ, et al. Modulation of BDNF cleavage by plasminogen-activator inhibitor-1 contributes to Alzheimer’s neuropathology and cognitive deficits. Biochim Biophys Acta 2017 Apr;1863(4):991-1001.
(37) Stratta P, Sanita P, Bonanni RL, de CS, Angelucci A, Rossi R, et al. Clinical correlates of plasma brain-derived neurotrophic factor in post-traumatic stress disorder spectrum after a natural disaster. Psychiatry Res 2016 Oct 30;244:165-70.
(38) Wentworth BA, Stein MB, Redwine LS, Xue Y, Taub PR, Clopton P, et al. Post-traumatic stress disorder: a fast track to premature cardiovascular disease? Cardiol Rev 2013 Jan;21(1):16-22.
(39) Vaughan DE. PAI-1 and atherothrombosis. J Thromb Haemost 2005 Aug;3(8):1879-83.
(40) Tsai SJ, Hong CJ, Liou YJ, Yu YW, Chen TJ. Plasminogen activator inhibitor-1 gene is associated with major depression and antidepressant treatment response. Pharmacogenet Genomics 2008 Oct;18(10):869-75.
(41) Jiang H, Li X, Chen S, Lu N, Yue Y, Liang J, et al. Plasminogen Activator Inhibitor-1 in depression: Results from Animal and Clinical Studies. Sci Rep 2016 Jul 26;6:30464.
(42) Teng HK, Teng KK, Lee R, Wright S, Tevar S, Almeida RD, et al. ProBDNF induces neuronal apoptosis via activation of a receptor complex of p75NTR and sortilin. J Neurosci 2005 Jun 1;25(22):5455-63.
(43) Woo NH, Teng HK, Siao CJ, Chiaruttini C, Pang PT, Milner TA, et al. Activation of p75(NTR) by proBDNF facilitates hippocampal long-term depression. Nat Neurosci 2005 Jul 17;8(8):1069-77.
(44) Yokoyama K, Hayashi M, Mogi C, Sasakawa Y, Watanabe G, Taya K, et al. Dose-dependent effects of a glucocorticoid on prolactin production. Endocr J 2008 May;55(2):405-14.
(45) Shi J, Wang L, Zhang H, Jie Q, Li X, Shi Q, et al. Glucocorticoids: Dose-related effects on osteoclast formation and function via reactive oxygen species and autophagy. Bone 2015 Oct;79:222-32.
(46) Descheemaeker KA, Wyns S, Nelles L, Auwerx J, Ny T, Collen D. Interaction of AP-1-, AP-2-, and Sp1-like proteins with two distinct sites in the upstream regulatory region of the plasminogen activator inhibitor-1 gene mediates the phorbol 12-myristate 13-acetate response. J Biol Chem 1992 Jul 25;267(21):15086-91.
(47) Petta I, Dejager L, Ballegeer M, Lievens S, Tavernier J, De BK, et al. The Interactome of the Glucocorticoid Receptor and Its Influence on the Actions of Glucocorticoids in Combatting Inflammatory and Infectious Diseases. Microbiol Mol Biol Rev 2016 Jun;80(2):495-522.
(48) Karin M, Chang L. AP-1--glucocorticoid receptor crosstalk taken to a higher level. J Endocrinol 2001 Jun;169(3):447-51.
(49) Bouarab C, Roullot-Lacarrière V, Vallée M et al. PAI-1 protein is a key molecular effector in the transition from normal to PTSD-like fear memory. Mol Psychiatry 2021. doi: 10.1038/s41380-021-01024-1. Online ahead of print. PMID: 33510345.

1. A method of preventing or treating a pathological conditions associated with intense stress in a patient in need thereof, comprising
administering to the patient a therapeutically effective amount of a PAI-1 antagonist, wherein said pathological conditions associated with intense stress is Post-Traumatic Stress Disorder (PTSD).

2. The method according to claim 1, wherein said PAI-1 antagonist directly binds to PAI-1 (protein or to a-nucleic sequence encoding PAI-1 and promotes tPA/plasmin activity to mediate the proteolytic processing of pro-BDNF to mature BDNF.

3. The method according to claim 1 wherein said PAI-1 antagonist is
1) an inhibitor of PAI-1 activity and/or
2) an inhibitor of PAI-1 gene expression.

4. The method according to claim 3 wherein said inhibitor of PAI-1 activity is selected from the group consisting of a small organic molecule, an anti-PAI-1 neutralizing antibody, a polypeptide and an aptamer.

5. The method according to claim 3 wherein the inhibitor of PAI-1 gene expression is selected from the group consisting of an antisense oligonucleotide, a nuclease, siRNA, shRNA and a ribozyme nucleic acid sequence.

6. A method for assessing a subject’s risk of having or developing pathological conditions associated with intense stress and treating the subject, said method comprising
measuring the level of PAI-1 protein in a body fluid sample obtained from said subject,
determining that the subject has a high level of PAI-1 protein compared to a control reference value, and
treating the subject determined to have a high level of PAI-1 protein compared to a control reference value by administering a PAI-1 antagonist,
wherein said pathological conditions associated with intense stress is Post-Traumatic Stress Disorder (PTSD).

7. The method according to claim 6 further comprising comparing said level of PAI-1 protein to a control reference value wherein:
a higher level of PAI-1 than the control reference value is predictive of a high risk of having or developing a Post-Traumatic Stress Disorder (PTSD) and
a lower level of PAI-1 than the control reference value is predictive of a low risk of having or developing a Post-Traumatic Stress Disorder (PTSD).

8. A method for monitoring the effect of a therapy for treating pathological conditions associated with intense stress in a subject and treating the subject comprising
measuring the level of PAI-1 in a first body fluid sample obtained from said subject at t1, wherein when t1 is prior to therapy,
measuring the level of PAI-1 in a second body fluid sample obtained from said subject at t2, wherein t2 is during or following therapy, and when t1 is during therapy, t2 is later during therapy or following therapy,
determining that the level of PAI-1 in the sample measured at t2 is decreased as compared to the level of PAI-1 in the sample measured at t1, and
treating the subject determined to have a high level of PAI-1 protein compared to a control reference value by administering the PAI-1 antagonist, wherein said pathological conditions associated with intense stress is Post-Traumatic Stress Disorder (PTSD).

9. The method according to claim 6, wherein the body fluid sample is blood sample and/or urine sample.

10. (canceled)

11. (canceled)

12. (canceled)

13. (canceled)


SEQUENCE LISTING (4 sequences)

SEQ ID NO: 1 - protein, 402 aa, Homo sapiens [sequence omitted]

SEQ ID NO: 2 - DNA, 3150 bp, Homo sapiens
acagctgtgtttggctgcagggccaagagcgctgtcaagaagacccacacgcccccctccagcagctgaattcctgcagctcagcagccgccgccagagcaggacgaaccgccaatcgcaaggcacctctgagaacttcaggatgcagatgtctccagccctcacctgcctagtcctgggcctggcccttgtctttggtgaagggtctgctgtgcaccatcccccatcctacgtggcccacctggcctcagacttcggggtgagggtgtttcagcaggtggcgcaggcctccaaggaccgcaacgtggttttctcaccctatggggtggcctcggtgttggccatgctccagctgacaacaggaggagaaacccagcagcagattcaagcagctatgggattcaagattgatgacaagggcatggcccccgccctccggcatctgtacaaggagctcatggggccatggaacaaggatgagatcagcaccacagacgcgatcttcgtccagcgggatctgaagctggtccagggcttcatgccccacttcttcaggctgttccggagcacggtcaagcaagtggacttttcagaggtggagagagccagattcatcatcaatgactgggtgaagacacacacaaaaggtatgatcagcaacttgcttgggaaaggagccgtggaccagctgacacggctggtgctggtgaatgccctctacttcaacggccagtggaagactcccttccccgactccagcacccaccgccgcctcttccacaaatcagacggcagcactgtctctgtgcccatgatggctcagaccaacaagttcaactatactgagttcaccacgcccgatggccattactacgacatcctggaactgccctaccacggggacaccctcagcatgttcattgctgccccttatgaaaaagaggtgcctctctctgccctcaccaacattctgagtgcccagctcatcagccactggaaaggcaacatgaccaggctgccccgcctcctggttctgcccaagttctccctggagactgaagtcgacctcaggaagcccctagagaacctgggaatgaccgacatgttcagacagtttcaggctgacttcacgagtctttcagaccaagagcctctccacgtcgcgcaggcgctgcagaaagtgaagatcgaggtgaacgagagtggcacggtggcctcctcatccacagctgtcatagtctcagcccgcatggcccccgaggagatcatcatggacagacccttcctctttgtggtccggcacaaccccacaggaacagtccttttcatgggccaagtgatggaaccctgaccctggggaaagacgccttcatctgggacaaaactggagatgcatcgggaaagaagaaactccgaagaaaagaattttagtgttaatgactctttctgaaggaagagaagacatttgccttttgttaaaagatggtaaaccagatctgtctccaagaccttggcctctccttggaggacctttaggtcaaactccctagtctccacctgagaccctgggagagaagtttgaagcacaactcccttaaggtctccaaaccagacggtgacgcctgcgggaccatctggggcacctgcttccacccgtctctctgcccactcgggtctgcagacctggttcccactgaggccctttgcaggatggaactacggggcttacaggagcttttgtgtgcctggtagaaactatttctgttccagtcacattgccatcactcttgtactgcctgccaccgcggaggaggctggtgacaggccaaaggccagtggaagaaacaccctttcatctcagagtccactgtggcactggccacccctccccagtacaggggtgctgcaggtggcagagtgaatgtcccccatcatgtggcccaactctcctggcctggccatctccctccccagaaacagtgtgcatgggttattttggagtgtaggtgacttgtttactcattgaagcagatttctgcttccttttatttttataggaatagaggaagaaatgtcagatgcgtgcccagctcttcaccccccaatctcttggtggggaggggtgtacctaaatatttatcatatccttgcccttgagtgcttgttagagagaaagagaactactaaggaaaataatattatttaaactcgctcctagtgtttctttgtggtctgtgtcaccgtatctcaggaagtccagccacttgactggcacacacccctccggacatccagcgtgacggagcccacactgccaccttgtggccgcctgagaccctcgcgccccccgcgcccctctttttccccttgatggaaattgaccatacaatttcatcctccttcaggggatcaaaaggacggagtggggggacagagactcagatgaggacagagtggtttccaatgtgttcaatagatttaggagcagaaatgcaaggggctgcatgacctaccaggacagaactttccccaattacagggtgactcacagccgcattggtgactcacttcaatgtgtcatttccggctgctgtgtgtgagcagtggacacgtgaggggggggtgggtgagagagacaggcagctcggattcaactaccttagataatatttctgaaaacctaccagccagagggtagggcacaaagatggatgtaatgcactttgggaggccaaggcgggaggattgcttgagcccaggagttcaagaccagcctgggcaacataccaagacccccgtctctttaaaaatatatatattttaaatatacttaaatatatatttctaatatctttaaatatatatatatattttaaagaccaatttatgggagaattgcacacagatgtgaaatgaatgtaatctaatagaagcctaatcagcccaccatgttctccactgaaaaatcctctttctttggggtttttctttctttcttttttgattttgcactggacggtgacgtcagccatgtacaggatccacaggggtggtgtcaaatgctattgaaattgtgttgaattgtatgctttttcacttttgataaataaacatgtaaaaatgtttcaaaaaaataataaaataaataaa

SEQ ID NO: 3 - DNA, 23 bp, Artificial
  Synthetic PAI-1 forward primer sequence
ggcacaatccaacagagacaatc

SEQ ID NO: 4 - DNA, 22 bp, Artificial
  Synthetic PAI-1 reverse primer sequence
aggcttctcatcccactctcaa